(19) United States
(12) Patent Application Publication
(10) Pub. No.: US 2021/0307628 A1
(43) Pub. Date: Oct. 7, 2021

(54) BLOOD PRESSURE MEASUREMENT DEVICE

(71) Applicants: OMRON Corporation, Kyoto (JP); OMRON HEALTHCARE Co., Ltd., Kyoto (JP)

(72) Inventors: Takashi ONO, Kyoto (JP); Takanori NISHIOKA, Kyoto (JP)

(21) Appl. No.: 17/304,492

(22) Filed: Jun. 22, 2021

Related U.S. Application Data
(63) Continuation of application No. PCT/JP2019/048027, filed on Dec. 9, 2019.

(30) Foreign Application Priority Data
Dec. 27, 2018 (JP) 2018-246072

(51) Int. Cl. A61B 5/022; A61B 5/00

(52) U.S. Cl. CPC A61B 5/02233; A61B 5/681

(57) ABSTRACT

A blood pressure measurement device includes a curler and a main body. The curler curves following a circumferential direction of a wrist from a hand back side of the wrist to a region where at least an artery is present on a hand palm side of the wrist through a first lateral side of the wrist. The curler includes a cover portion formed on the hand back side of the wrist and a member inserted into the cover portion. The device main body includes an outer case and a back cover. The outer case internally includes a pump and a flow path unit that supplies a fluid from the pump to a secondary side. The back cover is fixed to a first end portion of the outer case and the cover portion.

### CROSS-REFERENCE TO RELATED APPLICATIONS

This application is the U.S. national stage application filed pursuant to 35 U.S.C. 365(c) and 120 as a continuation of International Patent Application No. PCT/JP2019/048027, filed Dec. 9, 2019, which application claims priority to Japan Patent Application No. 2018-246072, Dec. 27, 2018, which applications are incorporated herein by reference in their entireties.

### TECHNICAL FIELD

The present invention relates to a blood pressure measurement device for measuring blood pressure.

### BACKGROUND ART

In recent years, blood pressure measurement devices for measuring blood pressure are being used to monitor health status at home, as well as in medical facilities. A blood pressure measurement device detects vibration of the artery wall to measure blood pressure by, for example, inflating and contracting a cuff wrapped around the upper arm or the wrist of a living body and detecting the pressure of the cuff using a pressure sensor.

A blood pressure measurement device referred to as, for example, a so-called integral type in which a cuff is integrated with a device main body supplying a fluid to the cuff has been known. In addition, as a technique for integrating a cuff and a device main body that supplies a fluid to the cuff, for example, as disclosed in JP 09-238910 A, there has been known a technique that provides members including, for example, claws and hooks between the cuff and a band, and fixes a blood pressure monitor body to the claws and the hooks (for example, see Patent Document 1).

### CITATION LIST

### Patent Literature

Patent Document 1: JP 09-238910 A

### SUMMARY OF INVENTION

### Technical Problem

In the blood pressure measurement device that fixes the device main body using the claws and the hooks described above, spaces for disposing the claws and the hooks are required, resulting in a large-sized device main body. Furthermore, in the configuration in which the device main body is fixed using the claws and the hooks, since holes for inserting the hooks and the claws through the device main body need to be provided, a waterproof property is possibly degraded. Especially, a wearable device attached to the wrist has been recently proposed, and there has been a demand for further downsizing and a waterproof property.

Thus, an object of the present invention is to provide a blood pressure measurement device that can be downsized and ensures a waterproof property.

### Solution to Problem

According to one aspect, there is provided a blood pressure measurement device attached to a wrist. The blood pressure measurement device includes a curler and a device main body. The curler curves following a circumferential direction of the wrist from a hand back side of the wrist to a region where at least an artery is present on a hand palm side of the wrist through a first lateral side of the wrist. The curler includes a cover portion formed on the hand back side of the wrist and an insert member for reinforcement inserted into the cover portion. The curler is made of a resin material. The device main body includes an outer case and a back cover. The outer case internally includes a pump and a flow path unit that supplies a fluid from the pump to a secondary side. The back cover is fixed to a first end portion of the outer case and the cover portion. A part of the back cover facing the cover portion is open. The back cover covers the first end portion of the outer case together with the cover portion.

According to this aspect, the cover portion that covers a back side of the outer case together with the back cover is provided on a portion of the curler, and the back cover is connected to the outer case and the cover portion. In addition, the insert member for reinforcement is provided on the cover portion. With the configurations, the curler and the outer case can be fixed via the back cover. This eliminates a need for providing, for example, a hook or a claw in the curler and eliminates a need for providing a hole through which the hook or the claw is inserted in the device main body, thus ensuring reducing dimensions in a thickness direction of the device main body. Moreover, while the curler is made of the resin material, the insert member for reinforcement is provided on the cover portion. This allows the cover portion to constitute a back lid covering an end portion of the outer case together with the back cover, and allows the cover portion to be reinforced by the insert member. Accordingly, compared to a case where the cover portion is made of only the resin material, a thickness can be reduced. As a result, a waterproof property of the blood pressure measurement device can be secured and downsizing of the blood pressure measurement device is possible.

In the blood pressure measurement device of the one aspect described above, there is provided a blood pressure measurement device that includes a first fastening member that fixes the back cover to the cover portion and a second fastening member that fixes the outer case and the back cover.

Here, the fastening members are members that mechanically fix two components, such as screws, bolts, small screws, and rivets.

According to this aspect, by fixing the back cover to the outer case and the cover portion with the fastening members, the back cover and the outer case, and the back cover and the cover portion can be easily fixed and disassembled, making manufacturing, replacement of the components, and maintenance easy. In addition, since the cover portion includes the insert member for reinforcement, joining strength of the first fastening member to the cover portion can be improved and the first fastening member can be downsized. As a result, in the blood pressure measurement device, the device main body is downsized and a degree of freedom of the design is improved.

In the blood pressure measurement device of the one aspect described above, there is provided a blood pressure measurement device in which the curler includes a relief portion through which the second fastening member is insertable at a position facing the second fastening member.

According to this aspect, by providing the relief portion at the position facing the second fastening member, which fixes the back cover and the outer case of the curler, the outer case can be fixed to the back cover using the second fastening member with the back cover attached to the cover portion of the curler. This facilitates assembly and disassembly of the blood pressure measurement device. Additionally, since the second fastening member can be provided at the position facing the curler, a degree of freedom of arrangement of the second fastening member is improved.

There is provided a blood pressure measurement device according to the one aspect of the blood pressure measurement device. The blood pressure measurement device includes a pressing cuff, a sensing cuff, and a tensile cuff. The pressing cuff includes a plurality of first bag-like structures, a first flow path body, and a first connection portion. The plurality of first bag-like structures are formed by joining two sheet members made of a resin material. The plurality of first bag-like structures are fixed to an inner circumferential surface on the hand palm side of the wrist of the curler and inflated by the fluid supplied from the pump. The first flow path body is integrally formed with the first bag-like structure by joining the two sheet members forming the first bag-like structure facing the curler. The first flow path body fluidly connects the pump and the first bag-like structure. The first flow path body has a distal end disposed on the wrist side of the cover portion. The first connection portion is provided at the distal end of the first flow path body. The first connection portion is inserted through the cover portion to be connected to the flow path unit. The sensing cuff includes a second bag-like structure, a second flow path body, and a second connection portion. The second bag-like structure is formed by joining the two sheet members. The second bag-like structure is fixed to a main surface on the wrist side of a back plate and inflated by the fluid supplied from the pump. The second flow path body is integrally formed with the second bag-like structure by joining the two sheet members forming the second bag-like structure. The second flow path body fluidly connects the pump and the second bag-like structure. The second flow path body has a distal end disposed on the wrist side of the cover portion. The second connection portion is provided at the distal end of the second flow path body. The second connection portion is inserted through the cover portion to be connected to the flow path unit. The tensile cuff includes a plurality of third bag-like structures and a third connection portion. The plurality of third bag-like structures are formed by joining the two sheet members. The plurality of third bag-like structures are fixed to an inner circumferential surface on the hand back side of the wrist of the curler and a position facing the cover portion and inflated by the fluid supplied from the pump. The third connection portion is provided at third bag-like structure facing the curler. The third connection portion is inserted through the cover portion to be connected to the flow path unit. The cover portion has respective three hole portions through which the first connection portion, the second connection portion, and the third connection portion are inserted.

Here, the fluid includes a liquid and air. The bag-like structure is inflated by the fluid and is an air bag when the fluid is air.

According to this aspect, the tensile cuff including the plurality of bag-like structures is disposed in a region where the cover portion of the curler is provided. Since the insert member for reinforcement is disposed on the cover portion, even when an external force is applied by expansion of the tensile cuff, strength of the curler is improved by the cover portion. As a result, deformation of the curler due to the expansion of the tensile cuff and creep deformation caused by repeated use can be suppressed. In addition, since the curler can maintain a predetermined shape, the blood pressure measurement device can suppress a decrease in measurement accuracy caused by repetitive blood pressure measurements.

In the blood pressure measurement device of the one aspect described above, there is provided a blood pressure measurement device in which the hole portion through which the second connection portion is inserted is disposed on a side where the sensing cuff of the cover portion is disposed with respect to the hole portions through which the first connection portion and the third connection portion are inserted.

According to this aspect, a length of the second flow path body that connects the second bag-like structure of the sensing cuff and the flow path unit of the device main body can be shorter than a length of the first flow path body that connects the first bag-like structure of the pressing cuff and the flow path unit of the device main body. This configuration allows reducing a volume of a region other than the bag-like structure for sensing in the sensing cuff as much as possible even in the configuration in which the device main body is disposed on the hand back side of the wrist and the sensing cuff is disposed on the hand palm side of the wrist. This allows the blood pressure measurement device to suitably detect blood pressure in blood pressure measurement by the sensing cuff, thereby ensuring improving blood pressure measurement accuracy.

In the blood pressure measurement device of the one aspect described above, there is provided a blood pressure measurement device in which the insert member is made of a metal material.

According to this aspect, by reinforcing the cover portion with the metal material having higher strength than the resin material, a thickness of the cover portion including the insert member can be reduced to the utmost extent, and joining strength with the fastening member can be improved. As a result, since the thickness of the cover portion can be thinned, the blood pressure measurement device can be downsized.

In the blood pressure measurement device according to the one aspect described above, there is provided a blood pressure measurement device in which the back cover and the cover portion mutually have flat abutment surfaces, and a sealing member is disposed between the back cover and the cover portion.

According to this aspect, by configuring the mutual abutment surfaces of the back cover and the cover portion to be flat and providing the sealing member between the abutment surfaces, a waterproof property between the back cover and the cover portion can be improved in a simple configuration.

### Advantageous Effects of Invention

The present invention can provide a blood pressure measurement device that can be downsized.

### BRIEF DESCRIPTION OF THE DRAWINGS

FIG. 1 is a perspective view illustrating a configuration of a blood pressure measurement device according to a first embodiment of the present invention.

FIG. 2 is an exploded perspective view illustrating the configuration of the blood pressure measurement device.

FIG. 3 is a side view illustrating the configuration of the blood pressure measurement device.

FIG. 4 is an explanatory diagram illustrating a state in which the blood pressure measurement device is attached to a wrist.

FIG. 5 is a block diagram illustrating the configuration of the blood pressure measurement device.

FIG. 6 is a perspective view illustrating the configuration of the blood pressure measurement device.

FIG. 7 is an exploded perspective view illustrating the configuration of the blood pressure measurement device.

FIG. 8 is an exploded perspective view illustrating the configuration of the blood pressure measurement device.

FIG. 9 is an exploded perspective view illustrating configurations of a curler and a cuff structure of the blood pressure measurement device.

FIG. 10 is a cross-sectional view illustrating the configurations of the curler and the cuff structure of the blood pressure measurement device.

FIG. 11 is a cross-sectional view illustrating the configurations of the curler and the cuff structure of the blood pressure measurement device.

FIG. 12 is a cross-sectional view illustrating a configuration of a tensile cuff of the blood pressure measurement device.

FIG. 13 is a cross-sectional view illustrating the configuration of the tensile cuff of the blood pressure measurement device.

FIG. 14 is a perspective view illustrating the configuration of the curler of the blood pressure measurement device.

FIG. 15 is a plan view illustrating the configuration of the cuff structure of the blood pressure measurement device.

FIG. 16 is a plan view illustrating the configuration of the cuff structure.

FIG. 17 is a plan view illustrating a configuration of a pressing cuff of the blood pressure measurement device.

FIG. 18 is a cross-sectional view illustrating the configuration of the pressing cuff.

FIG. 19 is a plan view illustrating a configuration of a sensing cuff of the blood pressure measurement device.

FIG. 20 is a cross-sectional view illustrating the configuration of the sensing cuff.

FIG. 21 is a flowchart depicting an example of a manufacturing method of the blood pressure measurement device.

FIG. 22 is a flowchart depicting an example of usage of the blood pressure measurement device.

FIG. 23 is a perspective view illustrating an example in which the blood pressure measurement device is attached to the wrist.

FIG. 24 is a perspective view illustrating an example in which the blood pressure measurement device is attached to the wrist.

FIG. 25 is a perspective view illustrating an example in which the blood pressure measurement device is attached to the wrist.

FIG. 26 is a cross-sectional view schematically illustrating a state in which the blood pressure measurement device is attached to a living body.

### DESCRIPTION OF EMBODIMENTS

### First Embodiment

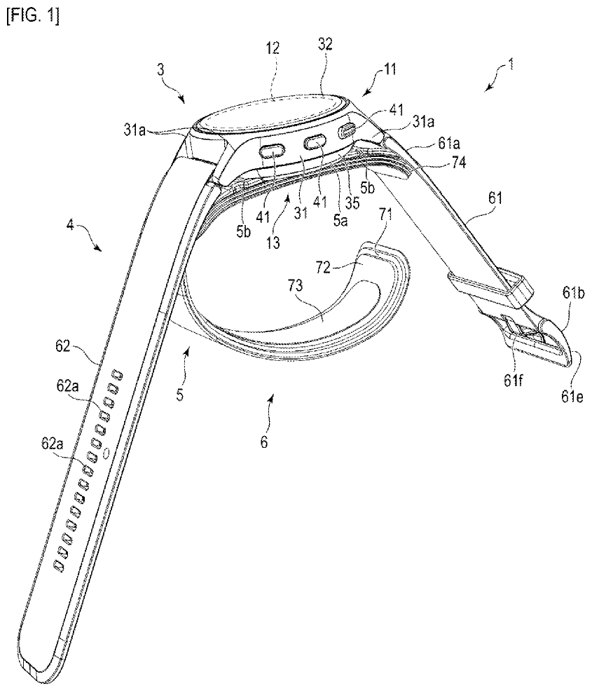

An example of a blood pressure measurement device 1 according to a first embodiment of the present invention will be exemplified below with reference to FIGS. 1 to 20.

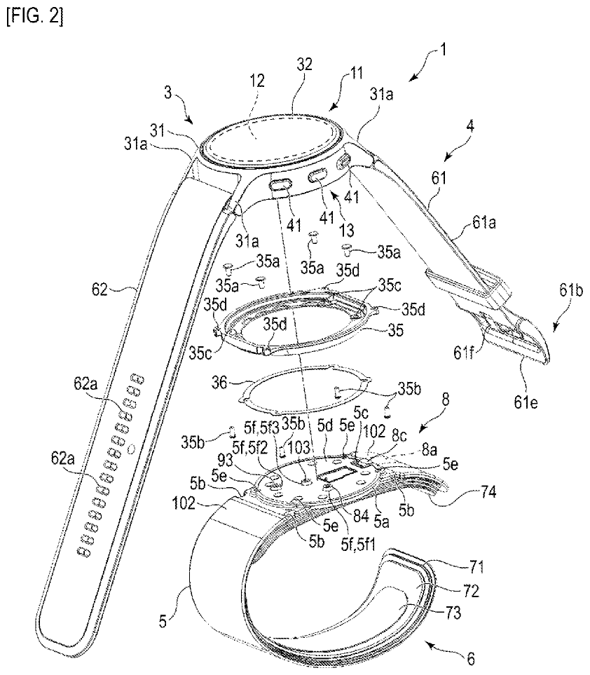

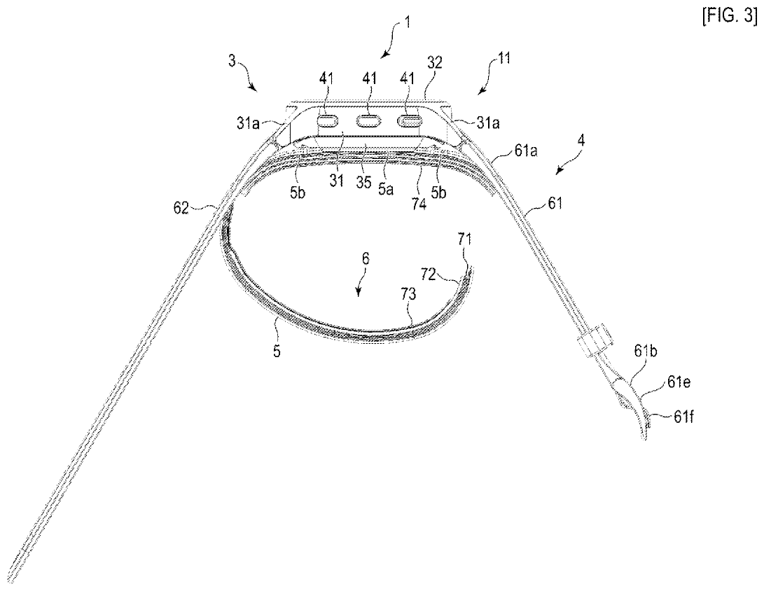

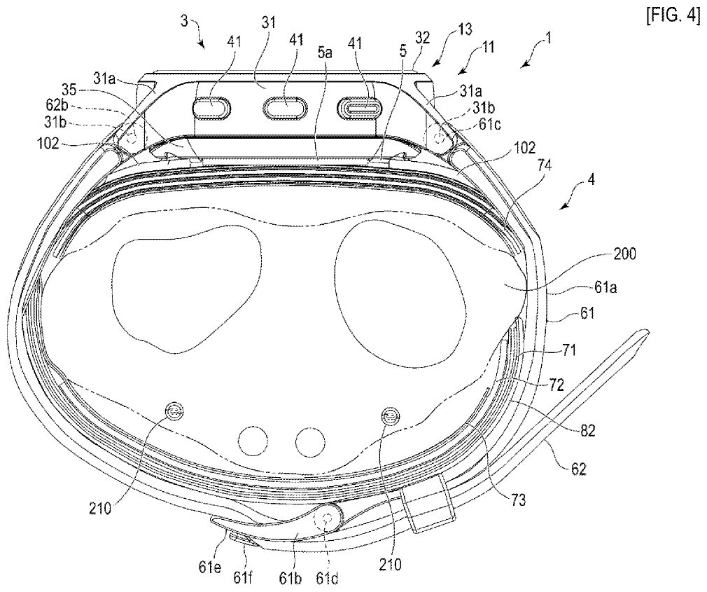

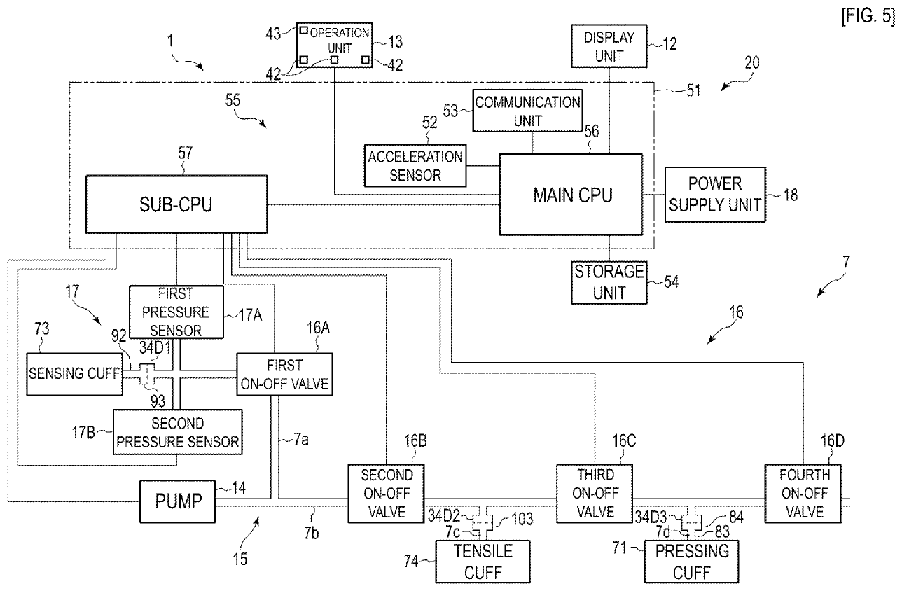

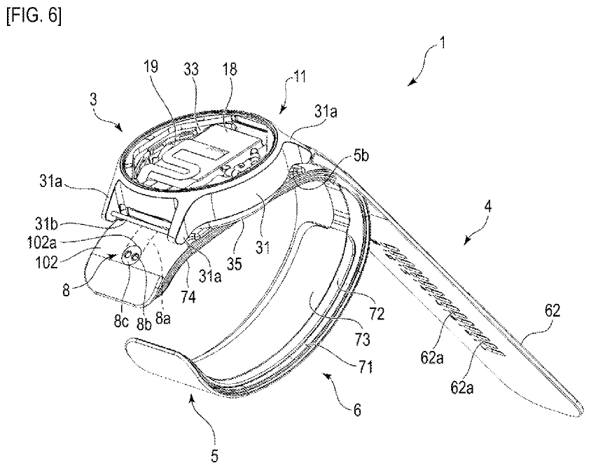

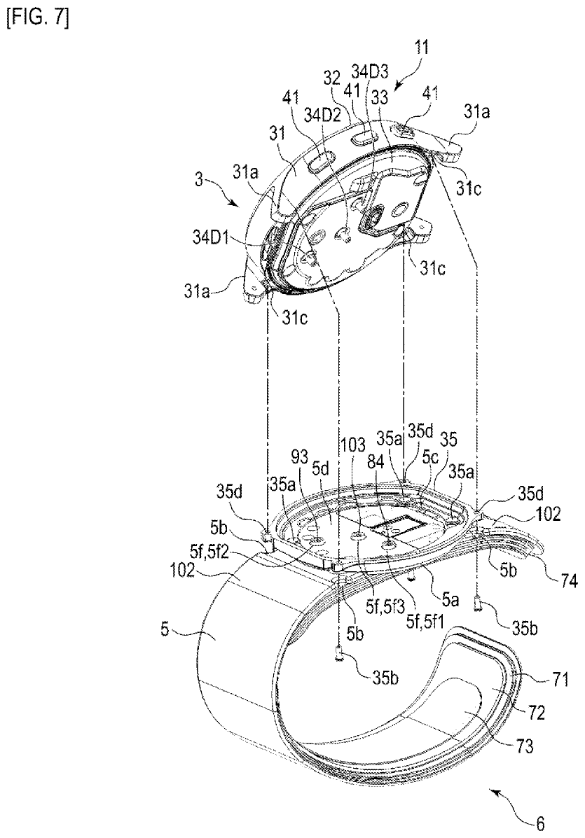

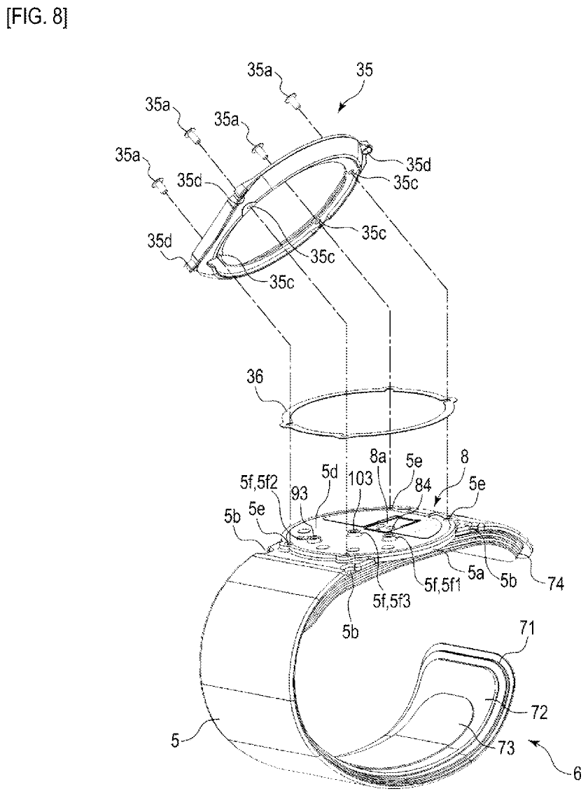

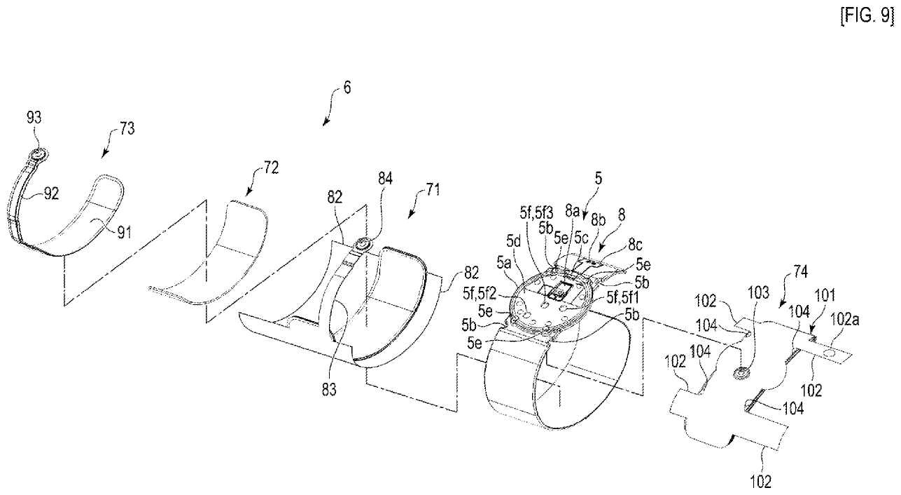

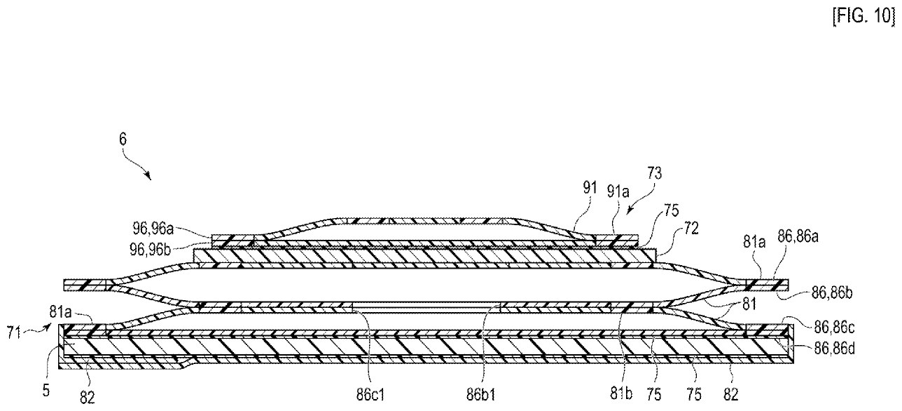

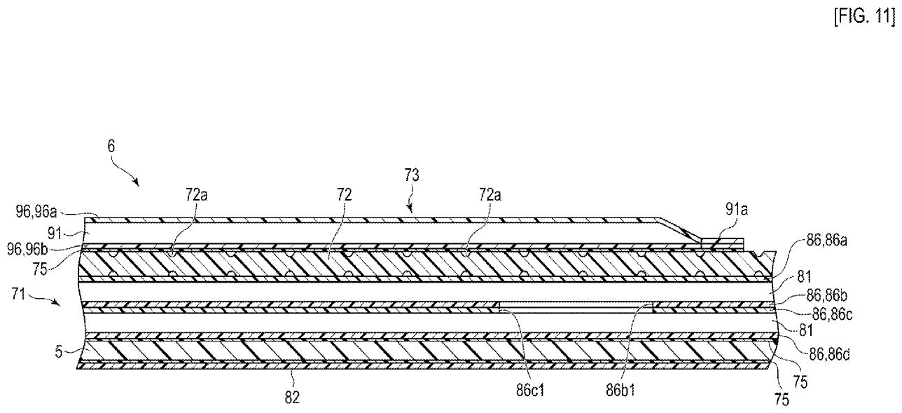

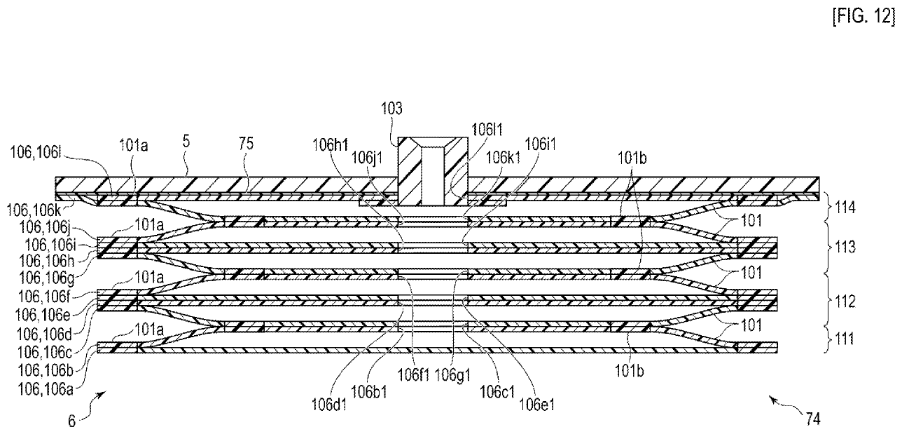

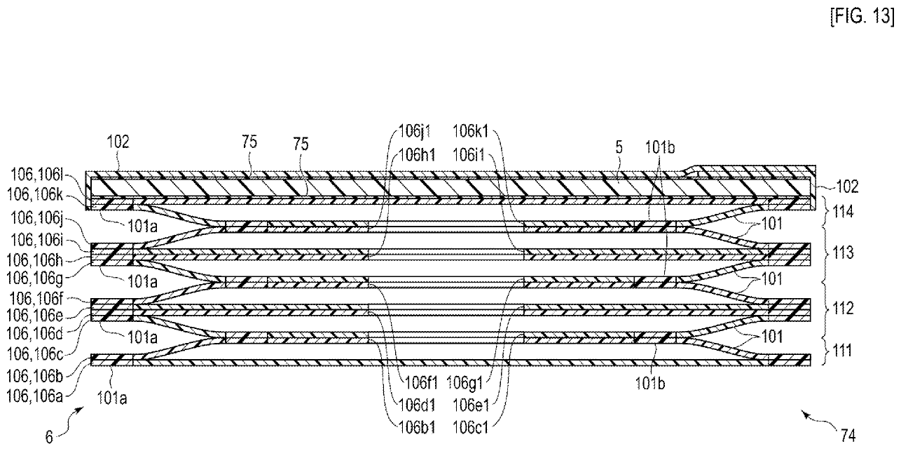

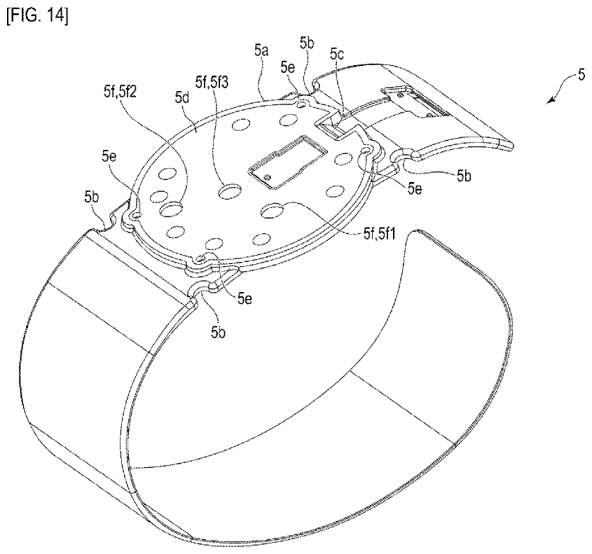

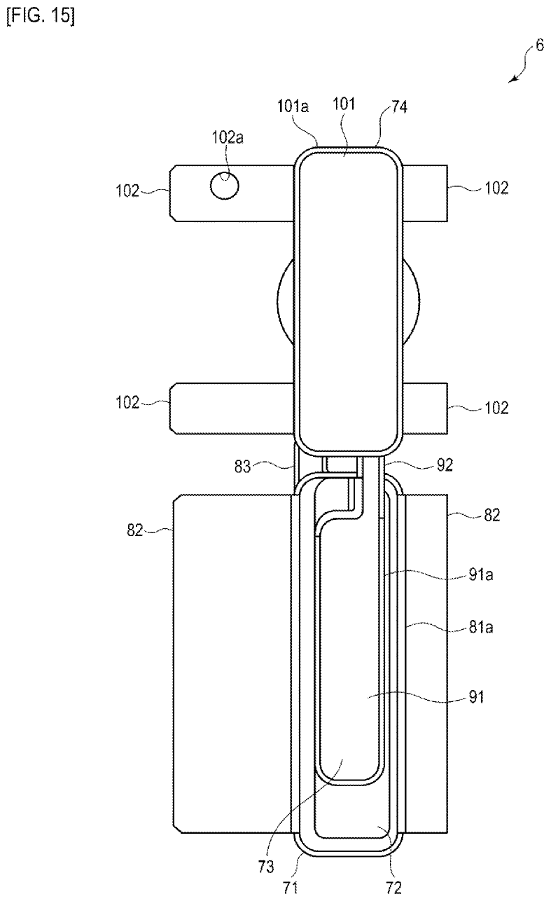

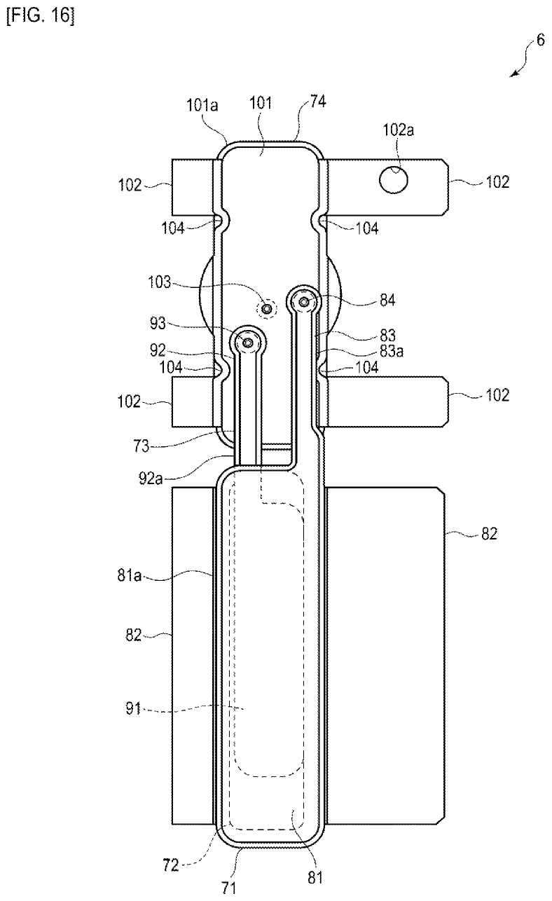

FIG. 1 is a perspective view illustrating a configuration of the blood pressure measurement device 1 according to the first embodiment of the present invention. FIG. 2 is an exploded perspective view illustrating the configuration of the blood pressure measurement device 1. FIG. 3 is a side view illustrating the configuration of the blood pressure measurement device 1. FIG. 4 is an explanatory diagram illustrating a state in which the blood pressure measurement device 1 is attached to a wrist 200. FIG. 5 is a block diagram illustrating the configuration of the blood pressure measurement device 1. FIG. 6 is a perspective view illustrating the blood pressure measurement device 1 in which a part of the configuration is omitted. FIG. 7 and FIG. 8 are exploded perspective views illustrating the blood pressure measurement device 1 in which a part of the configuration is omitted. FIG. 9 is an exploded perspective view illustrating configurations of a curler 5 and a cuff structure 6 of the blood pressure measurement device 1. FIG. 10 is a cross-sectional view illustrating the configurations of the curler 5 and the cuff structure 6 of the blood pressure measurement device 1. FIG. 11 is a cross-sectional view illustrating the configurations of the curler 5 and the cuff structure 6 of the blood pressure measurement device 1. FIG. 12 is a cross-sectional view illustrating a configuration of a tensile cuff 74 of the blood pressure measurement device 1. FIG. 13 is a cross-sectional view illustrating the configuration of the tensile cuff 74 of the blood pressure measurement device 1. FIG. 14 is a perspective view illustrating the configuration of the curler 5 of the blood pressure measurement device 1. FIG. 15 is a plan view illustrating the configuration of the cuff structure 6 on the wrist 200 side of the blood pressure measurement device 1. FIG. 16 is a plan view illustrating the configuration of the curler 5 on an inner circumferential surface side of the cuff structure 6.

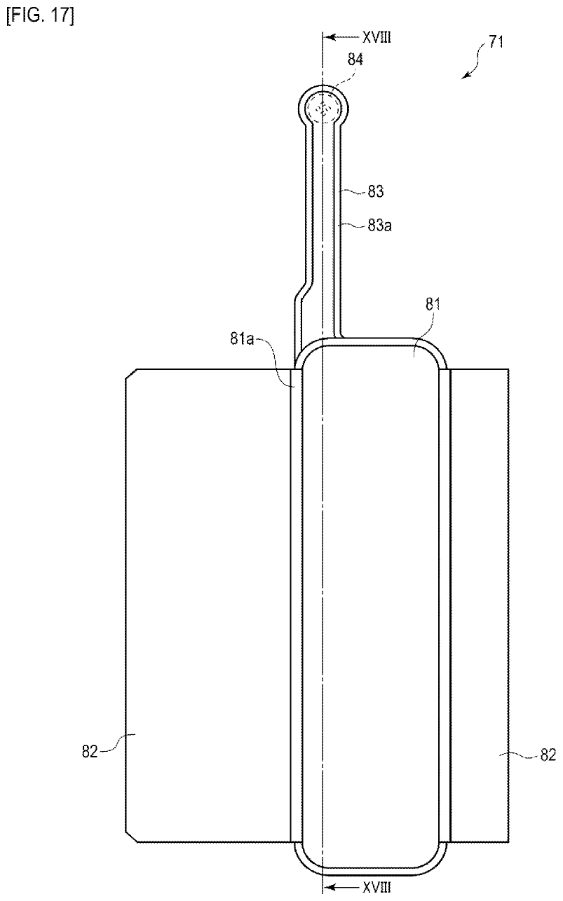

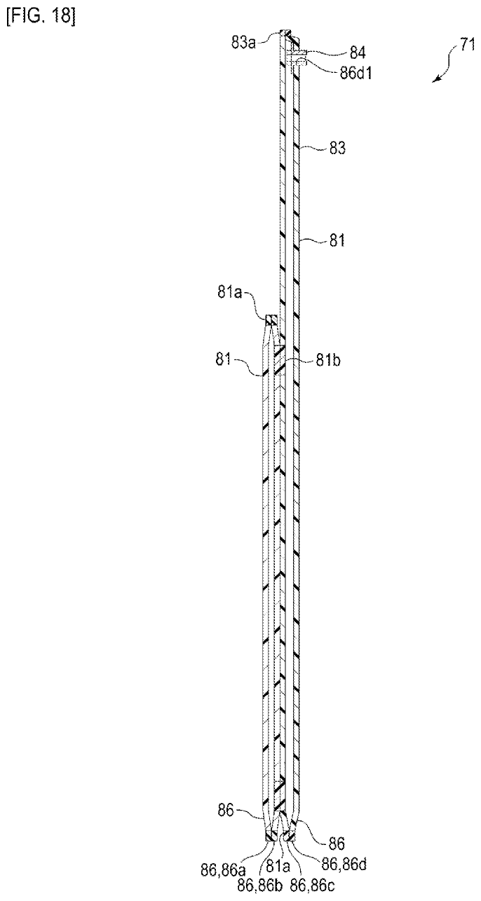

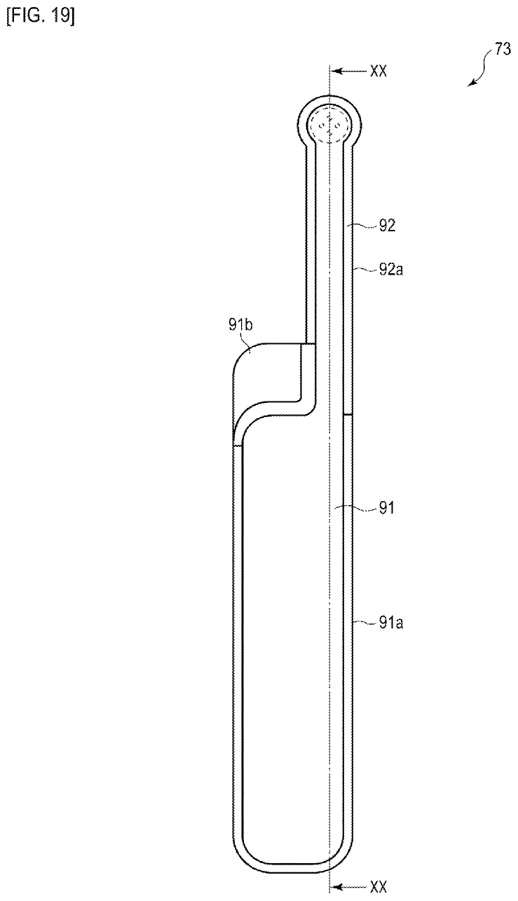

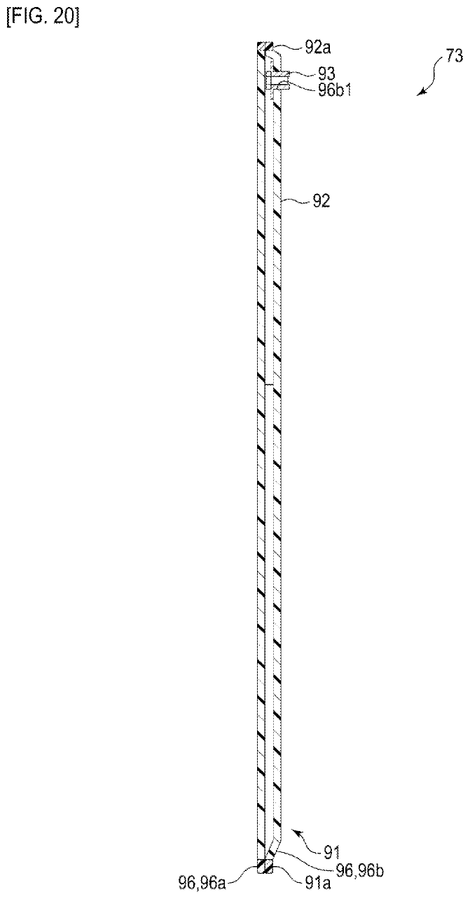

FIG. 17 is a plan view illustrating a configuration of a pressing cuff 71 of the blood pressure measurement device 1. FIG. 18 is a cross-sectional view illustrating the configuration of the pressing cuff 71 by a cross-sectional surface taken along the line XVIII-XVIII in FIG. 17. FIG. 19 is a plan view illustrating a configuration of a sensing cuff 73 of the blood pressure measurement device 1. FIG. 20 is a cross-sectional view illustrating the configuration of the sensing cuff 73 of the blood pressure measurement device 1 by a cross-sectional surface taken along the line XX-XX in FIG. 19.

The blood pressure measurement device 1 is an electronic blood pressure measurement device attached to a living body. The present embodiment will be described using an electronic blood pressure measurement device having an aspect of a wearable device attached to the wrist 200 of the living body.

As illustrated in FIGS. 1 to 6, the blood pressure measurement device 1 includes a device main body 3, a belt 4 that fixes the device main body 3 to the wrist, the curler 5 disposed between the belt 4 and the wrist, the cuff structure 6 including the pressing cuff 71, the sensing cuff 73, and the tensile cuff 74, a fluid circuit 7 fluidly connecting the device main body 3 and the cuff structure 6, and a power feed unit 8 provided in the curler 5.

As illustrated in FIGS. 1 to 6, the device main body 3 includes, for example, a case 11, a display unit 12, an operation unit 13, a pump 14, a flow path unit 15, an on-off valve 16, a pressure sensor 17, a power supply unit 18, a vibration motor 19, and a control substrate 20. The device main body 3 supplies a fluid to the cuff structure 6 using, for example, the pump 14, the on-off valve 16, the pressure sensor 17, and the control substrate 20.

As illustrated in FIGS. 1 to 3, the case 11 includes an outer case 31, a windshield 32 that covers an opening on a side (an outer side) opposite to the wrist 200 side of the outer case 31, a base 33 provided on the wrist 200 side inside the outer case 31, a back cover 35 that covers the wrist 200 side of the outer case 31, and a sealing member 36 provided on the lower surface of the back cover 35.

The outer case 31 is formed in a cylindrical shape. The outer case 31 includes pairs of lugs 31a provided at respective symmetrical positions in the circumferential direction of an outer circumferential surface, and spring rods 31b each provided between the lugs 31a of each of the two pairs of lugs 31a. As illustrated in FIG. 7, the outer case 31 has, for example, screw holes 31c at four locations in bases of the pair of lugs 31a, which are provided at the respective symmetrical positions in the circumferential direction of the outer circumferential surface on the surface (the rear surface) on the wrist 200 side. The windshield 32 is, for example, a circular glass plate.

The base 33 holds the display unit 12, the operation unit 13, the pump 14, the on-off valve 16, the pressure sensor 17, the power supply unit 18, the vibration motor 19, and the control substrate 20. Additionally, the base 33 constitutes a portion of the flow path unit 15 that makes the pump 14 and the cuff structure 6 fluidly continuous.

The back cover 35 is configured in an annular shape opening at the center side. The back cover 35 covers the outer peripheral edge side of the end portion on the wrist 200 side of the outer case 31. The back cover 35 is integrally combined with the curler 5 so that the center opening is covered with the curler 5, thus constituting a back lid that covers the end portion on the wrist 200 side of the outer case 31 together with the curler 5. Specifically, the back cover 35 is fixed to the curler 5 with four first fastening members 35a, and is fixed to the end portion on the wrist 200 side of the outer case 31 with four second fastening members 35b. The back cover 35 includes four hole portions 35c provided in the bottom portion through which the first fastening members 35a, which are fixed to the curler 5, are inserted. The back cover 35 radially protrudes at four locations of the outer circumferential portion, and four hole portions 35d through which the second fastening members 35b fixed to the outer case 31 are inserted are provided at the protruding parts. The surface of the back cover 35 that abuts on the curler 5 is configured to be a flat surface.

The first fastening member 35a and the second fastening member 35b are members that mechanically fix two components, such as screws, bolts, small screws, and rivets. In the present embodiment, the first fastening member 35a and the second fastening member 35b are screws.

As illustrated in FIG. 2 and FIG. 7, the four first fastening members 35a are screwed into screw holes 5e provided at four locations in a cover portion 5a, which will be described later, of the curler 5. The four second fastening members 35b are screwed into the screw holes 31c, which are provided at the four locations in the outer case 31.

As illustrated in FIGS. 2 and 8, the four hole portions 35c are provided in the bottom surface of the back cover 35, and are provided at positions facing the cover portion 5a, which will be described later, of the curler 5. For example, the four hole portions 35c have intervals therebetween in the extension direction of the curler 5 set to be smaller than intervals therebetween in a direction orthogonal to the extension direction of the curler 5.

As illustrated in FIGS. 2 and 7, the four hole portions 35d are provided at the protrusions in the outer circumferential portion of the back cover 35 so as to face the four locations of the bases of the pair of respective lugs 31a, which are provided at symmetrical positions in the circumferential direction of the outer circumferential surface on the surface (the rear surface) on the wrist 200 side of the outer case 31. When the outer case 31 is integrally combined with the back cover 35, the four hole portions 35d face the screw holes 31c in the outer case 31.

The sealing member 36 is, for example, a double-sided tape formed in a shape of a region where the back cover 35 contacts the curler 5. The sealing member 36 is provided between abutment surfaces of the curler 5 and the back cover 35 where the curler 5 and the back cover 35 mutually abut on to seal between the curler 5 and the back cover 35.

The display unit 12 is disposed on the base 33 of the outer case 31 and directly below the windshield 32. As illustrated in FIG. 5, the display unit 12 is electrically connected to the control substrate 20. The display unit 12 is, for example, a liquid crystal display or an organic electroluminescence display. The display unit 12 displays various types of information including the date and time and measurement results of blood pressure values such as the systolic blood pressure and diastolic blood pressure, heart rate, and the like.

The operation unit 13 is configured to be capable of receiving an instruction input from a user. For example, as illustrated in FIGS. 1 and 5, the operation unit 13 includes a plurality of buttons 41 provided on the case 11, a sensor 42 that detects operations of the buttons 41, and a touch panel 43 provided on the display unit 12 or the windshield 32. When operated by the user, the operation unit 13 converts an instruction into an electrical signal. The sensor 42 and the touch panel 43 are electrically connected to the control substrate 20 to output electrical signals to the control substrate 20.

As the plurality of buttons 41, for example, three buttons are provided. The buttons 41 are supported by the base 33 and protrude from the outer circumferential surface of the outer case 31. The plurality of buttons 41 and a plurality of the sensors 42 are supported by the base 33. The touch panel 43 is integrally provided on the windshield 32, for example.

The pump 14 is, for example, a piezoelectric pump. The pump 14 compresses air and feeds compressed air to the cuff structure 6 through the flow path unit 15. The pump 14 is electrically connected to the control substrate 20.

As illustrated in FIG. 5, the flow path unit 15 constitutes a flow path connecting from the pump 14 to the pressing cuff 71 and the tensile cuff 74 and a flow path connecting from the pump 14 to the sensing cuff 73. Additionally, the flow path unit 15 constitutes a flow path connecting from the pressing cuff 71 and the tensile cuff 74 to the atmosphere, and a flow path connecting from the sensing cuff 73 to the atmosphere. The flow path unit 15 is a flow path of air constituted by, for example, a hollow portion, a groove, a flow path tank, and a tube, provided in, for example, the base 33.

The on-off valve 16 opens and closes a portion of the flow path unit 15. A plurality of the on-off valves 16, as a specific example, the four on-off valves 16 as illustrated in FIG. 5, are provided and selectively open and close the flow path connecting from the pump 14 to the pressing cuff 71 and the tensile cuff 74, the flow path connecting from the pump 14 to the sensing cuff 73, the flow path connecting from the pressing cuff 71 and the tensile cuff 74 to the atmosphere, and the flow path connecting from the sensing cuff 73 to the atmosphere, by the combination of opening and closing of each of the on-off valves 16. As a specific example, the four on-off valves 16 are configured by a first on-off valve 16A, a second on-off valve 16B, a third on-off valve 16C, and a fourth on-off valve 16D. The first on-off valve 16A opens and closes the flow path connecting the pump 14 and the sensing cuff 73. The second on-off valve 16B opens and closes the flow path connecting the pump 14 and the tensile cuff 74. The second on-off valve 16B and the third on-off valve 16C open and close the flow path connecting the pump 14 and the pressing cuff 71. The second on-off valve 16B, the third on-off valve 16C, and the fourth on-off valve 16D open and close the flow path connecting the pump 14 and the atmosphere.

The pressure sensor 17 at least detects the pressure in the sensing cuff 73. The pressure sensor 17 includes, for example, a first pressure sensor 17A and a second pressure sensor 17B. The pressure sensor 17 converts a detected pressure into an electrical signal, and outputs the electrical signal to the control substrate 20. For example, the first pressure sensor 17A and the second pressure sensor 17B are provided in the flow path of the flow path unit 15 connecting the first on-off valve 16A and the sensing cuff 73. The pressing cuff 71, the sensing cuff 73, the tensile cuff 74, and the pump 14 are continuous in these flow paths by opening and closing the respective on-off valves, and thus the pressures in these flow paths corresponds to the pressures in the internal spaces of the pressing cuff 71, the sensing cuff 73, and the tensile cuff 74 to which the pump 14 is connected.

As a specific example, when the first on-off valve 16A is open and the second on-off valve 16B is closed, the pressure sensor 17 detects the pressure in the sensing cuff 73, in other words, the pressure in the flow path unit 15 connecting the pump 14 and the sensing cuff 73. Additionally, when the first on-off valve 16A and the second on-off valve 16B are open and the third on-off valve 16C is closed, the pressure sensor 17 detects the pressure in the sensing cuff 73 and the tensile cuff 74, in other words, the pressure in the flow path unit 15 connecting the pump 14, the sensing cuff 73, and the tensile cuff 74. Furthermore, when the first on-off valve 16A, the second on-off valve 16B, and the third on-off valve 16C are open and the fourth on-off valve 16D is open or closed, the pressure sensor 17 detects the pressure in the pressing cuff 71, the sensing cuff 73, and the tensile cuff 74, in other words, the pressure in the flow path unit 15 connecting the pump 14, the pressing cuff 71, the sensing cuff 73, and the tensile cuff 74.

The power supply unit 18 is, for example, a secondary battery such as a lithium ion battery. As illustrated in FIG. 5, the power supply unit 18 is electrically connected to the control substrate 20. The power supply unit 18 supplies power to the control substrate 20.

As illustrated in FIG. 5, the control substrate 20 includes, for example, a substrate 51, an acceleration sensor 52, a communication unit 53, a storage unit 54, and a control unit 55. The control substrate 20 is constituted by the acceleration sensor 52, the communication unit 53, the storage unit 54, and the control unit 55 that are mounted on the substrate 51.

The substrate 51 is fixed to the base 33 of the case 11 using screws or the like.

The acceleration sensor 52 is, for example, a 3-axis acceleration sensor. The acceleration sensor 52 outputs, to the control unit 55, an acceleration signal representing acceleration of the device main body 3 in three directions orthogonal to each other. For example, the acceleration sensor 52 is used to measure, from the detected acceleration, the amount of activity of a living body to which the blood pressure measurement device 1 is attached.

The communication unit 53 is configured to be able to transmit and receive information to and from an external device wirelessly or by wire. For example, the communication unit 53 transmits information controlled by the control unit 55, and information of a measured blood pressure value, a pulse, and the like to an external device via a network, and receives a program or the like for software update from an external device via a network and sends the program or the like to the control unit 55.

In the present embodiment, the network is, for example, the Internet, but is not limited to this. The network may be a network such as a Local Area Network (LAN) provided in a hospital or may be direct communication with an external device using a cable or the like including a terminal of a predetermined standard such as a USB. Thus, the communication unit 53 may be configured to include a plurality of wireless antennas, micro-USB connectors, or the like.

The storage unit 54 pre-stores program data for controlling the overall blood pressure measurement device 1 and the fluid circuit 7, settings data for setting various functions of the blood pressure measurement device 1, calculation data for calculating a blood pressure value and a pulse from pressure measured by the pressure sensors 17, and the like. Additionally, the storage unit 54 stores information such as a measured blood pressure value and a measured pulse.

The control unit 55 is constituted by one or more CPUs, and controls operation of the overall blood pressure measurement device 1 and operation of the fluid circuit 7. The control unit 55 is electrically connected to and supplies power to the display unit 12, the operation unit 13, the pump 14, each of the on-off valves 16 and the pressure sensors 17. Additionally, the control unit 55 controls operation of the display unit 12, the pump 14, and the on-off valves 16, based on electrical signals output by the operation unit 13 and the pressure sensors 17.

For example, as illustrated in FIG. 5, the control unit 55 includes a main Central Processing Unit (CPU) 56 that controls operation of the overall blood pressure measurement device 1, and a sub-CPU 57 that controls operation of the fluid circuit 7. For example, the main CPU 56 obtains measurement results such as blood pressure values, for example, the systolic blood pressure and the diastolic blood pressure, and the heart rate, from electrical signals output by the pressure sensor 17, and outputs an image signal corresponding to the measurement results to the display unit 12.

For example, the sub-CPU 57 drives the pump 14 and the on-off valves 16 to feed compressed air to the pressing cuff 71 and the sensing cuff 73 when an instruction to measure the blood pressure is input from the operation unit 13. In addition, the sub-CPU 57 controls driving and stopping of the pump 14 and opening and closing of the on-off valves 16 based on electrical signal output by the pressure sensors 17. The sub-CPU 57 controls the pump 14 and the on-off valves 16 to selectively feed compressed air to the pressing cuff 71 and the sensing cuff 73 and selectively depressurize the pressing cuff 71 and the sensing cuff 73.

As illustrated in FIGS. 1 to 4 and 6, the belt 4 includes a first belt 61 provided on the first pair of lugs 31a and the first spring rod 31b, and a second belt 62 provided on the second pair of lugs 31a and the second spring rod 31b. The belt 4 is wrapped around the wrist 200 with the curler 5 in between.

The first belt 61 is referred to as a so-called a parent and is configured like a band that can be coupled to the second belt 62. As illustrated in FIGS. 1 to 3, the first belt 61 includes a belt portion 61a and a buckle 61b. The belt portion 61a is configured as a band-like shape. The belt portion 61a is made of an elastically deformable resin material. In addition, the belt portion 61a is flexible and internally includes a sheet-like insert member that suppresses longitudinal expansion and contraction of the belt portion 61a. The belt portion 61a includes a first hole portion 61c formed in the first end portion and orthogonal to the longitudinal direction of the belt portion 61a and a second hole portion 61d formed in the second end portion and orthogonal to the longitudinal direction of the first belt 61.

As illustrated in FIGS. 4 and 6, the first hole portion 61c is provided in the end portion of the belt portion 61a. The first hole portion 61c has an inner diameter at which the spring rod 31b can be inserted into the first hole portion 61c and at which the first belt 61 can rotate with respect to the spring rod 31b. In other words, the first belt 61 is rotatably held by the outer case 31 by disposing the first hole portion 61c between the pair of lugs 31a and around the spring rod 31b.

As illustrated in FIGS. 1 and 3, the second hole portion 61d is provided at the distal end of the belt portion 61a. The buckle 61b is attached to the second hole portion 61d.

As illustrated in FIGS. 1 and 3, the buckle 61b includes a frame body 61e in a rectangular frame shape and a prong 61f rotatably attached to the frame body 61e. A side of the frame body 61e to which the prong 61f is attached is inserted into the second hole portion 61d, and the frame body 61e is attached rotatably with respect to the belt portion 61a.

The second belt 62 is referred to as a so-called blade tip, and is configured in a band-like shape having a width at which the second belt 62 can be inserted into the frame body 61e. The second belt 62 is made of an elastically deformable resin material. In addition, the second belt 62 is flexible and internally includes a sheet-like insert member that suppresses longitudinal expansion and contraction of the second belt 62.

In addition, as illustrated in FIGS. 1, 2, and 6, the second belt 62 includes a plurality of small holes 62a into which the prong 61f is inserted. Additionally, the second belt 62 includes a third hole portion 62b provided at first end portion of the second belt 62 and extending orthogonally to the longitudinal direction of the second belt 62. The third hole portion 62b has an inner diameter at which the spring rod 31b can be inserted into the third hole portion 62b and at which the second belt 62 can rotate with respect to the spring rod 31b. In other words, the second belt 62 is rotatably held by the outer case 31 by disposing the third hole portion 62b between the pair of lugs 31a and around the spring rod 31b.

Thus, the second belt 62 of the belt 4 is inserted into the frame body 61e, and the prong 61f is inserted into the small hole 62a in the belt 4, the first belt 61 and the second belt 62 are integrally connected together, and the belt 4, together with the outer case 31, comes to have an annular shape following the wrist 200 along the circumferential direction. The belt 4 has the annular shape following the circumferential direction of the wrist 200, thereby pressing the curler 5 and elastically deforming the curler 5 such that the curler 5 follows the circumferential direction of a wrist of a wearer of the blood pressure measurement device 1.

As illustrated in FIGS. 1 to 4, the curler 5 is configured in a band-like shape that curves in such a manner as to follow along the circumferential direction of the wrist 200. The curler 5 is formed with a first end and a second end spaced apart from each other. For example, a first end-side outer surface of the curler 5 is fixed to the back cover 35 of the device main body 3. The first end and the second end of the curler 5 are disposed at positions where the first end and the second end protrude to the first lateral side of the wrist 200 further than the back cover 35. Accordingly, the curler 5 has the first end and the second end disposed on the lateral side of the wrist 200 when the blood pressure measurement device 1 is attached to the wrist 200. Furthermore, the first end and the second end of the curler 5 are located adjacent to each other at a predetermined distance from each other. The curler 5 is made of a resin material, for example. As a specific example, the curler 5 is made of polypropylene with a thickness of approximately 1 mm.

As illustrated in FIGS. 1 to 4, as a specific example, the curler 5 is configured in a band-like shape that curves in such a manner as to follow along the circumferential direction of the wrist. Furthermore, the curler 5 includes the circular plate-shaped cover portion 5a provided at a position facing the hand back side of the wrist 200 on the first end side and constituting a back lid together with the back cover 35, and relief portions 5b provided around the cover portion 5a to which the second fastening members 35b, which fix the outer case 31 and the back cover 35, can be moved. For example, in the curler 5, the cover portion 5a and the adjacent portions are formed in a flat plate shape, and the first end side and the second end side with respect to the cover portion 5a are formed so as to curve at a predetermined curvature. A length of the curler 5 from the cover portion 5a to the first end is formed shorter than a length from the cover portion 5a to the second end. As a specific example, in the curler 5, the short side from the cover portion 5a to the first end is disposed on the hand back side of the wrist, and the long side from the cover portion 5a to the second end extends from the hand back side of the wrist to the hand palm side of the wrist 200 through one lateral side.

As illustrated in FIGS. 3, 4, and 14, the curler 5 is formed in a shape such that when the first end and the second end of the curler 5 are close, the second end is positioned on the inner circumferential surface side of the first end. As a specific example, a width in the width direction of the wrist 200 of the curler 5 is set such that the hand back side of the wrist 200 of the curler 5 becomes greater than that of the hand palm side of the wrist 200 of the curler 5. Furthermore, a radius of curvature of the first end on the hand back side of the wrist 200 of the curler 5 is set greater than a radius of curvature of the second end on the hand palm side of the wrist 200. With the configuration, in the curler 5, when both end sides of the curler 5 abut on, the second end is disposed more inward of the curler 5 than the first end. Further, the curler 5 includes a recess 5c provided adjacent to the cover portion 5a in a part of the cover portion 5a and the outer surface on the first end side of the cover portion 5a, and in other words, the outer surface on the short side extending from the cover portion 5a.

The cover portion 5a includes an insert member 5d for reinforcement that is inserted. The cover portion 5a is fixed to the wrist 200 side of the outer case 31 via the fixed back cover 35. The cover portion 5a has the screw holes 5e provided at positions facing the four hole portions 35c in the back cover 35 and into which the first fastening members 35a to fix the back cover 35 are screwed, and three hole portions 5f for connecting the cuff structure 6 to the device main body 3. The surface of the cover portion 5a that abuts on the back cover 35 is set to a flat surface.

When the back cover 35 is fixed to the outer case 31 from the back cover 35 side with the second fastening members 35b, the relief portions 5b serve as a relief to dispose the second fastening members 35b on the back cover 35 without an interference of the second fastening members 35b to the curler 5 and dispose tools to rotate the second fastening members 35b.

As illustrated in FIG. 14, the insert member 5d is a thin plate formed in the same shape as the main surface shape of the cover portion 5a or a shape slightly smaller than the main surface shape of the cover portion 5a. The insert member 5d is made of a material having a higher bending strength than that of the curler 5, for example, a metal material. As a specific example, the insert member 5d is made of a SUS material.

The three hole portions 5f are a first hole portion 5f1 formed to have an inner diameter into which a connection portion 84 described later of the pressing cuff 71 can be inserted, a second hole portion 5f2 formed to have an inner diameter into which a connection portion 93 described later of the sensing cuff 73 can be inserted, and a third hole portion 5f3 formed to have an inner diameter into which a connection portion 103 described later of the tensile cuff 74 can be inserted. In the present embodiment, the second hole portion 5f2 is disposed closer to the second end side of the hand palm side in the cover portion 5a of the curler 5 than the first hole portion 5f1 and the third hole portion 5f3.

The curler 5 is fixed to the outer case 31 in the direction in which the first end and the second end face the second belt 62 of the belt 4. In the curler 5, at least a position facing the hand palm side of the wrist 200 curves along the circumferential direction along the hand palm side of the wrist 200 to hold the cuff structure 6 facing the hand palm side of the wrist 200 in a curved state following the shape of the hand palm side of the wrist 200.

The curler 5 has hardness to provide flexibility and shape retainability. Here, “flexibility” refers to deformation of the shape of the curler 5 in a radial direction at the time of application of an external force of the belt 4 to the curler 5. For example, “flexibility” refers to deformation of the shape of the curler 5 in a side view in which the curler 5 approaches the wrist, is along the shape of the wrist, or follows to the shape of the wrist when the curler 5 is pressed by the belt 4. Furthermore, “shape retainability” refers to the ability of the curler 5 to maintain a pre-imparted shape when no external force is applied to the curler 5. For example, “shape retainability” refers to, in the present embodiment, the ability of the curler 5 to maintain the shape in a shape curving along the circumferential direction of the wrist.

The cuff structure 6 is disposed on an inner circumferential surface of the curler 5, and is held along the shape of the inner circumferential surface of the curler 5. As a specific example, the curler 5 holds the cuff structure 6 by disposing the pressing cuff 71 and the tensile cuff 74 on the inner circumferential surface and fixing the cuff structure 6 with bonding layers 75 provided between the curler 5, the pressing cuff 71, and the tensile cuff 74. In the present embodiment, the bonding layer 75 is an adhesive or a double-sided tape.

As illustrated in FIGS. 1 to 9, 15, and 16, the cuff structure 6 includes the pressing cuff 71, a back plate 72, the sensing cuff 73, and the tensile cuff 74. The cuff structure 6 also includes the bonding layers 75 that bond the respective configurations and the curler 5 and the cuffs 71, 74 together. The cuff structure 6 is fixed to the curler 5. The cuff structure 6 includes the pressing cuff 71, the back plate 72, and the sensing cuff 73 that are stacked each other and disposed on the curler 5, and the tensile cuff 74 that is spaced apart from the pressing cuff 71, the back plate 72, and the sensing cuff 73 and disposed on the curler 5.

As a specific example, as illustrated in FIG. 4, the cuff structure 6 is stacked on the inner circumferential surface on the hand palm side of the wrist 200 of the curler 5 with the pressing cuff 71, the back plate 72, and the sensing cuff 73 in this order from the inner circumferential surface of the curler 5 toward the wrist 200 side. In addition, the cuff structure 6 includes the tensile cuff 74 disposed on the inner circumferential surface on the hand back side of the wrist 200 of the curler 5. Each of the members of the cuff structure 6 is fixed to an adjacent member of the cuff structure 6 in a stacking direction with the bonding layer 75.

The pressing cuff 71 is fluidly connected to the pump 14 through the flow path unit 15. The pressing cuff 71 is inflated to press the back plate 72 and the sensing cuff 73 toward the wrist 200 side. As illustrated in FIGS. 10, 11, and 15 to 18, the pressing cuff 71 includes a plurality of, for example, two layers of air bags 81, joined portions 82 provided on the air bag 81 facing the curler 5, a flow path body (a first flow path body) 83 in communication with the air bag 81, and the connection portion (a first connection portion) 84 provided on the distal end of the flow path body 83. The pressing cuff 71 is configured by integrally welding a plurality of sheet members 86.

Here, the air bag 81 are bag-like structure (first bag-like structures), and in the present embodiment, the blood pressure measurement device 1 is configured to use air with the pump 14, and thus the present embodiment will be described using the air bags. However, in a case where a fluid other than air is used, the bag-like structures only need to be fluid bags inflated by the fluid. The plurality of air bags 81 are stacked and are in fluid communication with one another in the stacking direction.

The air bag 81 is formed in a rectangular bag shape that is long in one direction. Additionally, the air bag 81 has a width in the short direction set to be the same width as a width in the short direction of the curler 5. The air bag 81 is constituted, for example, by combining the two sheet members 86 and thermally welding the sheet members 86 in a rectangular frame shape long in one direction, like a welded portion 81a illustrated in FIGS. 10, 11, and 15 to 18. In addition, the two layers of the air bags 81 are formed by thermally welding the two air bags 81 and combining them integrally, or welding the facing sheet members 86 of the adjacent air bags 81 and then welding the air bags 81, thus forming the air bags 81. As a specific example, the two layers of the air bags 81 are fluidly continuous with openings provided in the sheet members 86 facing each other. In addition, in the two layers of the air bags 81, bridge welding is performed on the facing sheet members 86 so as to have a quadrilateral frame shape smaller than the welded portions 81a located on the outer peripheral edges, and surrounding the plurality of openings with the bridge welded portions (the joining portions) 81b to integrally form the adjacent air bags 81, and thus the air bags 81 are fluidly connected inside the bridge welded portions 81b. Here, the bridge welding and the bridge of the bridge welded portions 81b mean integral joining of the adjacent air bags 81.

One or a plurality of the joined portions 82 are provided on at least a part of an edge portion of the air bag 81 disposed adjacent to the curler 5. The joined portion 82 is formed by a part of the sheet member 86 constituting the air bag 81.

In the present embodiment, as illustrated in FIGS. 10, 11, and 15 to 18, an example in which one joined portion 82 is provided at each edge portion in the short direction of the air bag 81 will be described. Note that, for example, the joined portions 82 may be divided in the longitudinal direction of the air bag 81 by a slit, or a plurality of the joined portions 82 may be provided in the longitudinal direction of the air bag 81. The joined portions 82 are joined to at least the outer circumferential surface of the curler 5 when the pressing cuff 71 is disposed on the inner circumferential surface of the curler 5. Furthermore, for example, the two joined portions 82 are stacked and welded.

Note that the two joined portions 82 are set, for example, to have lengths that differ in the short direction of the air bag 81. In this example, the two joined portions 82 are stacked and welded on the first end side in the short direction of the curler 5. As long as the distal ends can be disposed on the outer circumferential surface of the curler 5, the lengths of the two joined portions 82 can be appropriately set, and may be or need not to be stackable. However, when the lengths of the joined portions 82 are set to be stackable, the lengths are preferably set such that the distal ends do not extend outward of the outer edges of the outer circumferential surface of the curler 5.

As illustrated in FIGS. 9 and 15 to 18, the flow path body 83 is integrally provided on one air bag 81, for example, a part of a first edge portion in the longitudinal direction of the air bag 81 adjacent to the curler 5. As a specific example, the flow path body 83 is provided at an end portion of the air bag 81 near the device main body 3. Additionally, the flow path body 83 is formed in a shape that is long in one direction with a width smaller than the width in the short direction of the air bag 81, and its distal end is formed in a circular shape. The flow path body 83 includes the connection portion 84 at the distal end. The connection portion 84 is, for example, a nipple. As illustrated in FIGS. 5 and 7, the flow path body 83 is connected to the flow path unit 15 by inserting a third nozzle 34D3 provided in the device main body 3 into the connection portion 84, thus constituting a flow path between the flow path unit 15 of the device main body 3 and the air bag 81.

The flow path body 83 is configured by thermally welding a part of the sheet members 86 adjacent to the region constituting the air bag 81 of the sheet members 86 in a frame shape long in one direction with the connection portion 84 disposed in the two sheet members 86. The flow path body 83 is disposed between the inner circumferential surface of the curler 5 and the tensile cuff 74, and its distal end is disposed on the main surface on the wrist 200 side of the region where the cover portion 5a of the curler 5 is provided and at a position facing the first hole portion 5f1. In addition, a width of the flow path body 83 excluding a welded portion 83a is formed to, for example, 3.8 mm.

Note that the air bag 81 in which the flow path body 83 is provided is configured by configuring a part of the welded portions 81a to weld the two sheet members 86 in the rectangular frame shape in a non-welded state such that the air bag 81 is continuous with the welded portion 83a constituting the flow path body 83, and thus the air bag 81 is fluidly continuous with the flow path body 83.

The connection portion 84 is provided at the distal end of the flow path body 83. The distal end of the connection portion 84 is exposed from the sheet member 86 facing the curler 5 among the two sheet members 86 constituting the flow path body 83. The connection portion 84 is inserted through the first hole portion 5f1 of the cover portion 5a and is connected to the flow path unit 15.

As a specific example, as illustrated in FIGS. 10, 11, and 26, the pressing cuff 71 includes, from the wrist 200 side, a first sheet member 86a, a second sheet member 86b, a third sheet member 86c, and a fourth sheet member 86d. The second sheet member 86b constitutes a first-layer air bag 81 along with the first sheet member 86a, the third sheet member 86c is integrally joined to the second sheet member 86b and constitutes the joined portions 82, and the fourth sheet member 86d constitutes the second-layer air bag 81 and the flow path body 83 with the third sheet member 86c. Note that the pressing cuff 71 is integrally constituted by joining the adjacent sheet members 86 by thermal welding.

The first sheet member 86a and the second sheet member 86b are formed to have the rectangular shape similar to the air bag 81, and the peripheral edge portions of the four sides are welded to constitute the air bag 81. The second sheet member 86b and the third sheet member 86c are disposed facing each other, and include a plurality of openings 86b1 and 86c1, respectively, through which the two air bags 81 are fluidly continuous. Additionally, the second sheet member 86b and the third sheet member 86c are integrally joined by thermally welding the peripheries of the plurality of openings 86b1, 86c1 in a quadrilateral frame shape smaller than the four sides where the air bag 81 is welded.

The third sheet member 86c is configured in a shape so as to ensure configuring the air bag 81, the joined portions 82, and the flow path body 83, for example. The fourth sheet member 86d is configured in a shape so as to ensure configuring the air bag 81 and the flow path body 83, for example. Furthermore, the fourth sheet member 86d has a hole portion 86d1 into which the distal end of the connection portion 84 can be inserted, for example.

The third sheet member 86c and the fourth sheet member 86d are disposed so as to face each other and are thermally welded along the peripheral edge shapes of the air bag 81 and the flow path body 83 so that the air bag 81 and the flow path body 83 are fluidly continuous, and cut into predetermined shapes to constitute the air bag 81, the joined portions 82, and the flow path body 83.

The connection portion 84 is disposed in the hole portion 86d1 of the fourth sheet member 86d, and the periphery of the hole portion 86d1 is thermally welded with the connection portion 84. Further, each of the fourth sheet member 86d is bonded to the inner circumferential surface of the curler 5 and the joined portion 82 of the third sheet member 86c is bonded to the outer circumferential surface of the curler 5 via the bonding layers 75.

As illustrated in FIGS. 10, 11, and 26, the back plate 72 is stuck to the outer surface of the first sheet member 86a of the pressing cuff 71 with the bonding layer 75. The back plate 72 is formed in a plate shape using a resin material. The back plate 72 is made of polypropylene, for example, and is formed into a plate shape having a thickness of approximately 1 mm. The back plate 72 has shape followability.

Here, “shape followability” refers to a function of the back plate 72 by which the back plate 72 can be deformed in such a manner as to follow the shape of a contacted portion of the wrist 200 to be disposed, and the contacted portion of the wrist 200 refers to a region of the wrist 200 that is faced by the back plate 72. The contact here includes both direct contact and indirect contact via the sensing cuff 73.

For example, as illustrated in FIG. 11, the back plate 72 includes a plurality of grooves 72a in both main surfaces of the back plate 72 and extending in a direction orthogonal to the longitudinal direction. The plurality of respective grooves 72a face each other in the thickness direction of the back plate 72. Additionally, the plurality of grooves 72a are disposed at equal intervals in the longitudinal direction of the back plate 72.

In the back plate 72, portions including the plurality of grooves 72a are thinner than portions including no grooves 72a and thus the portions including the plurality of grooves 72a are easily deformed. Accordingly, the back plate 72 is deformed in such a manner as to follow to the shape of the wrist 200, and has shape followability of extending in the circumferential direction of the wrist. The back plate 72 is formed such that the length of the back plate 72 is sufficient to cover the hand palm side of the wrist 200. The back plate 72 transfers the pressing force from the pressing cuff 71 to the main surface on the back plate 72 side of the sensing cuff 73 in a state in which the back plate 72 runs along the shape of the wrist 200.

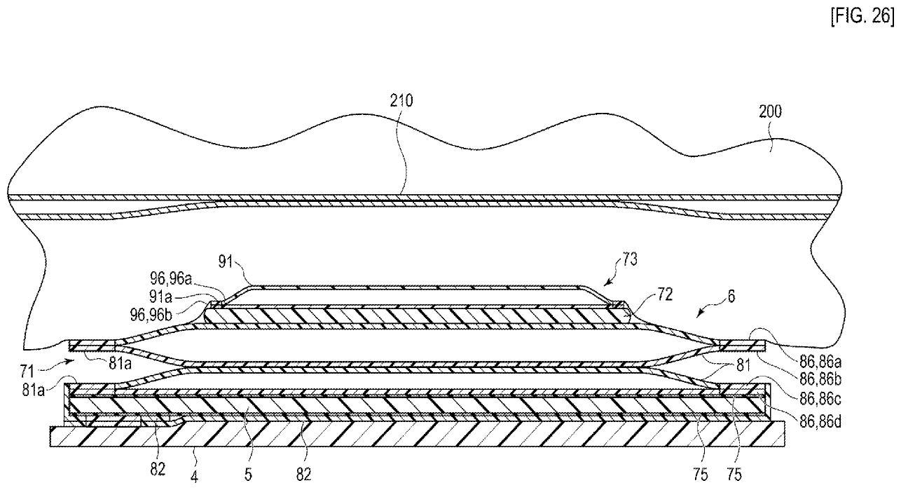

The sensing cuff 73 is fluidly connected to the pump 14 through the flow path unit 15. The sensing cuff 73 is fixed to the main surface on the wrist 200 side of the back plate 72. As illustrated in FIGS. 4 and 26, the sensing cuff 73 is in direct contact with a region of the wrist 200 where an artery 210 is present. The artery 210 as used herein is the radial artery and the ulnar artery. The sensing cuff 73 is formed in the same shape as that of the back plate 72 or a shape that is smaller than that of the back plate 72, in the longitudinal direction and the width direction of the back plate 72. The sensing cuff 73 is inflated to compress a hand palm-side region of the wrist 200 in which the artery 210 resides. The sensing cuff 73 is pressed by the inflated pressing cuff 71 toward the wrist 200 side with the back plate 72 in between.

As a specific example, as illustrated in FIGS. 10, 11, 19, and 20, the sensing cuff 73 includes one air bag 91, a flow path body (a second flow path body) 92 in communication with the air bag 91, and a connection portion (a second connection portion) 93 provided on a distal end of a flow path body 92. One main surface of the air bag 91 of the sensing cuff 73 is fixed to the back plate 72. For example, the sensing cuff 73 is bonded to the main surface on the wrist 200 side of the back plate 72 with the bonding layer 75. The sensing cuff 73 is configured by integrally welding a plurality of sheet members 96.

Here, the air bag 91 is a bag-like structure (a second bag-like structure), and in the present embodiment, the blood pressure measurement device 1 is configured to use air with the pump 14, and thus the present embodiment will be described using the air bag. However, in a case where a fluid other than air is used, the bag-like structure may be, for example, a liquid bag.

The air bag 91 is constituted in a rectangular shape that is long in one direction. The air bag 91 is constituted, for example, by combining the two sheet members 96 that are long in one direction and thermally welding the sheet members 96 in a rectangular frame shape long in one direction, like a welded portion 91a illustrated in FIGS. 10, 11, 19, and 20. The air bag 91 includes, for example, a bonding margin 91b to ensure an area for bonding the air bag 91 to the back plate 72 using the bonding layer 75. The bonding margin 91b is formed by, for example, the sheet member 96 facing the back plate 72.

The flow path body 92 is integrally provided on a part of a first edge portion in the longitudinal direction of the air bag 91. As a specific example, the flow path body 92 is provided at an end portion of the air bag 91 near the device main body 3. Additionally, the flow path body 92 is formed in a shape that is long in one direction with a width smaller than the width in the short direction of the air bag 91, and its distal end is formed in a circular shape. The flow path body 92 includes the connection portion 93 at the distal end. The connection portion 93 is, for example, a nipple. As illustrated in FIGS. 5 and 7, the flow path body 92 is connected to the flow path unit 15 by inserting a first nozzle 34D1 provided in the device main body 3 into the connection portion 93, thus constituting a flow path between the flow path unit 15 of the device main body 3 and the air bag 91.

The flow path body 92 is configured by thermally welding a part of the sheet members 96 adjacent to the region constituting the air bag 91 of the sheet members 96 in a frame shape long in one direction with the connection portion 93 disposed in the two sheet members 96. Note that the air bag 91 is configured by configuring a part of the welded portions 91a to weld the two sheet members 96 in the rectangular frame shape in a non-welded state such that the air bag 91 is continuous with a welded portion 92a constituting the flow path body 92, and thus the air bag 91 is fluidly continuous with the flow path body 92. The flow path body 92 is disposed between the inner circumferential surface of the curler 5 and the tensile cuff 74, and its distal end is disposed on the main surface on the wrist 200 side of the region where the cover portion 5a of the curler 5 is provided and at a position facing the second hole portion 5f2. In addition, a width of the flow path body 92 excluding the welded portion 92a is formed to, for example, 3.8 mm.

The connection portion 93 is provided at the distal end of the flow path body 92. The distal end of the connection portion 93 is exposed outside from the sheet member 96 facing the curler 5 and the back plate 72 among the two sheet members 96 constituting the flow path body 92. The connection portion 93 is inserted through the second hole portion 5f2 of the cover portion 5a and is connected to the flow path unit 15.

As a specific example, as illustrated in FIGS. 10 and 11, the sensing cuff 73 includes a fifth sheet member 96a and a sixth sheet member 96b from the wrist 200 side. Note that the sensing cuff 73 is constituted by joining the adjacent sheet members 96 by thermal welding.

The fifth sheet member 96a and the sixth sheet member 96b are configured in a shape so as to ensure configuring the air bag 91, the bonding margin 91b, and the flow path body 92, for example. The fifth sheet member 96 and the sixth sheet member 96b are disposed so as to face each other and are thermally welded along the peripheral edge shapes of the air bag 91 and the flow path body 92 so that the air bag 91 and the flow path body 92 are fluidly continuous, and cut into predetermined shapes to constitute the air bag 91 and the flow path body 92.

Furthermore, the sixth sheet member 96b has a hole portion 96b1 into which the distal end of the connection portion 93 can be inserted, for example. The connection portion 93 is disposed in the hole portion 96b1, and the periphery of the hole portion 96b1 is thermally welded with the connection portion 93. The sixth sheet member 96b is bonded to the inner circumferential surface of the back plate 72 via the bonding layer 75.

The tensile cuff 74 is fluidly connected to the pump 14 through the flow path unit 15. The tensile cuff 74 is inflated to press the curler 5 such that the curler 5 is spaced apart from the wrist 200, thus pulling the belt 4 and the curler 5 toward the hand back side of the wrist 200. The tensile cuff 74 includes a plurality of, for example, six layers of air bags 101, joined portions 102 provided on the air bag 101 facing the curler 5, the connection portion 103 provided on the air bag 101 facing the curler 5, and notch portions 104 provided in at least the air bag 101 facing the curler 5. The tensile cuff 74 is configured by integrally welding a plurality of sheet members 106. The tensile cuff 74 is fixed to a region where the flow path bodies 83, 92 are provided, and the hand back side of the wrist 200 of the curler 5 including the cover portion 5a. That is, the flow path body 83 of the pressing cuff 71 and the flow path body 92 of the sensing cuff 73 are disposed between the hand back side of the wrist 200 of the curler 5 and the tensile cuff 74.

Additionally, the tensile cuff 74 is configured such that the thickness of the tensile cuff 74 in an inflating direction, in the present embodiment, in the direction in which the curler 5 and the wrist 200 face each other, during inflation, is larger than the thickness of the pressing cuff 71 in the inflating direction during inflation and larger than the thickness of the sensing cuff 73 in the inflating direction during inflation. That is, the air bags 101 of the tensile cuff 74 have more layer structures than the air bags 81 in the pressing cuff 71 and the air bag 91 in the sensing cuff 73, and are thicker than the pressing cuff 71 and the sensing cuff 73 when the air bags 101 are inflated from the curler 5 toward the wrist 200.

In the present embodiment, the tensile cuff 74 including the six layers of the air bags 101 includes a first outer layer 111 constituted by one air bag 101, a first intermediate layer 112 constituted by the two layers of the air bags 101 that are thermally welded to the first outer layer 111 to be integrally combined, a second intermediate layer 113 constituted by the two layers of the air bags 101 that are thermally welded to the first intermediate layer 112 to be integrally combined, and a second outer layer 114 constituted by one air bag 101 that is thermally welded to the second intermediate layer 113 to be integrally combined.

Here, the air bags 101 are bag-like structures (third bag-like structures), and in the present embodiment, the blood pressure measurement device 1 is configured to use air with the pump 14, and thus the present embodiment will be described using the air bags. However, in a case where a fluid other than air is used, the bag-like structures only need to be fluid bags inflated by the fluid. A plurality of the air bags 101 are stacked and are in fluid communication in the stacking direction.

The air bag 101 is formed in a rectangular bag shape that is long in one direction. Additionally, the air bag 101 has a width in the short direction set to be the same width as a width in the short direction of the curler 5. The air bag 101 is constituted, for example, by combining the two sheet members 106 and thermally welding the sheet members 106 in a rectangular frame shape long in one direction, like a welded portion 101a illustrated in FIGS. 12, 13, 15, and 16. The six layers of the air bags 101 are fluidly continuous with openings provided in the sheet members 106 facing each other.

In addition, in the six layers of the air bags 101, bridge welding is performed on the sheet members 106 where the first outer layer 111 and the first intermediate layer 112, the first intermediate layer 112 and the second intermediate layer 113, and the second intermediate layer 113 and the second outer layer 114 each face each other so as to have a quadrilateral frame shape smaller than the welded portions 81a located on the outer peripheral edges, and surrounding the plurality of openings with bridge welded portions (joining portions) 101b to integrally form the adjacent air bags 101, and thus the air bags 101 are fluidly connected inside the bridge welded portions 101b.

The first outer layer 111 is formed by one air bag 101 disposed on the wrist 200 side. The first outer layer 111 constitutes the air bag 101 of the first layer from the wrist 200 side among the six layers of the air bags 101.

The first intermediate layer 112 is stacked on the first outer layer 111. The first intermediate layer 112 is formed by the two layers of the air bags 101. The first intermediate layer 112 constitutes the air bags 101 of the second layer and the third layer from the wrist 200 side among the six layers of the air bags 101. The first intermediate layer 112 is constituted by integrally welding the two layers of the air bags 101 at the outer peripheral edges. In other words, the first intermediate layer 112 is formed by integrally welding the four sheet members 106 in the outer peripheral edge shapes of the air bags 101.

The second intermediate layer 113 is stacked on the first intermediate layer 112. The second intermediate layer 113 is formed of the two layers of the air bags 101. The second intermediate layer 113 constitutes the air bags 101 of the fourth layer and the fifth layer from the wrist 200 side among the six layers of the air bags 101. The second intermediate layer 113 is constituted by integrally welding the two layers of the air bags 101 at the outer peripheral edges. In other words, the second intermediate layer 113 is formed by integrally welding the four sheet members 106 in the outer peripheral edge shapes of the air bags 101.

The second outer layer 114 is formed by one air bag 101 disposed on the curler 5 side. The second outer layer 114 constitutes the air bag 101 of the sixth layer from the wrist 200 side among the six layers of the air bags 101.

One or a plurality of the joined portions 102 are provided on at least a part of an edge portion of the air bag (the air bag of the sixth layer) 101 disposed adjacent to the curler 5. The joined portion 102 is formed by a part of the sheet member 106 constituting the air bag 101.

In the present embodiment, an example in which the two joined portions 102 are provided at each edge portion in the short direction of the air bag 101 in the longitudinal direction of the air bag 101 will be described. Note that, for example, the joined portions 102 are provided on the air bag 101 avoiding a position facing the cover portion 5a of the curler 5. Further, for example, the joined portion 102 includes a relief portion 102a at a position facing power feed terminals 8b, which will be described later, of the power feed unit 8 provided in the curler 5, to expose the power feed terminals 8b to the outside. The relief portion 102a is, for example, an opening that can expose the power feed terminals 8b to the outside, and has a circular shape as an example.

The joined portions 102 are joined to at least the outer circumferential surface of the curler 5 when the tensile cuff 74 is disposed on the inner circumferential surface of the curler 5. Additionally, the joined portions 102 disposed at the same position in the short direction of the air bags 101 are stacked and welded.

Note that the two joined portions 102 are set, for example, to have lengths that differ in the short direction of the air bag 101. In this example, the two joined portions 102 are stacked and welded on the first end side in the short direction of the curler 5. As long as the distal ends can be disposed on the outer circumferential surface of the curler 5, the lengths of the two joined portions 102 can be appropriately set, and may be or need not to be stackable. However, when the lengths of the joined portions 102 are set to be stackable, the lengths are preferably set such that the distal ends do not extend outward of the outer edges of the outer circumferential surface of the curler 5.

The connection portion 103 is, for example, a nipple. The connection portion 103 is disposed on the center side in the longitudinal direction of the air bag 101 disposed adjacent to the curler 5 and at a position facing the third hole portion 5f3 in the cover portion 5a. The distal end of the connection portion 103 is exposed from the sheet member 106 facing the curler 5 among the two sheet members 106 constituting the air bag 101. As illustrated in FIGS. 5 and 7, the connection portion 103 is connected to the flow path unit 15 by being connected to a second nozzle 34D2 provided in the device main body 3.

The notch portions 104 are provided at positions facing the relief portions 5b provided on the curler 5. The notch portions 104 are provided in the air bag 101 of the sixth layer that forms the second outer layer 114.

As a specific example, as illustrated in FIGS. 12 and 13, the tensile cuff 74 includes a seventh sheet member 106a, an eighth sheet member 106b, a ninth sheet member 106c, a tenth sheet member 106d, an eleventh sheet member 106e, a twelfth sheet member 106f, a thirteenth sheet member 106 g, a fourteenth sheet member 106h, a fifteenth sheet member 106i, a sixteenth sheet member 106j, a seventeenth sheet member 106k, and an eighteenth sheet member 106l in this order from the wrist 200 side. Note that the tensile cuff 74 is integrally constituted by joining the adjacent sheet members 106 by thermal welding.

The seventh sheet member 106a to the eighteenth sheet member 106l are configured in a rectangular shape similar to the air bag 101. The seventh sheet member 106a and the eighth sheet member 106b are thermally welded along the shapes of the peripheral edge portions of the four sides of the air bag 101 to constitute the air bag 101 of the first layer (the first layer) from the wrist 200 side. That is, the seventh sheet member 106a and the eighth sheet member 106b constitute the first outer layer 111.

The eighth sheet member 106b and the ninth sheet member 106c are disposed facing each other, and include a plurality of openings 106b1, 106c1, respectively, through which the two air bags 101 are fluidly continuous. Additionally, the eighth sheet member 106b and the ninth sheet member 106c are integrally joined by bridge welding by heat on the peripheries of the plurality of openings 106b1, 106c1 in a quadrilateral frame shape smaller than the four sides where the air bag 101 is welded.

The ninth sheet member 106c and the tenth sheet member 106d are thermally welded along the shapes of the peripheral edge portions of the four sides of the air bag 101 to constitute the air bag 101 of the second layer (the second layer) from the wrist 200 side.

As illustrated in FIGS. 12 and 13, the tenth sheet member 106d and the eleventh sheet member 106e are disposed facing each other, and include a plurality of openings 106d1, 106e1, respectively, through which the two air bags 101 are fluidly continuous. The eleventh sheet member 106e and the twelfth sheet member 106f are thermally welded along the shapes of the peripheral edge portions of the four sides of the air bag 101 to constitute the air bag 101 of the third layer (the third layer) from the wrist 200 side.

Note that the ninth sheet member 106c, the tenth sheet member 106d, the eleventh sheet member 106e, and the twelfth sheet member 106f are integrally and thermally welded along the shapes of the peripheral edge portions of the four sides of the air bags 101 to constitute the first intermediate layer 112 in which the air bags 101 of the second layer and the third layer are integrally formed.

As illustrated in FIGS. 12 and 13, the twelfth sheet member 106f and the thirteenth sheet member 106g are disposed facing each other, and include a plurality of openings 106f1, 106g1, respectively, through which the two air bags 101 are fluidly continuous. Additionally, the twelfth sheet member 106f and the thirteenth sheet member 106g are integrally joined by bridge welding by heat on the peripheries of the plurality of openings 106f1, 106g1 in a quadrilateral frame shape smaller than the four sides where the air bag 101 is welded.

The thirteenth sheet member 106g and the fourteenth sheet member 106h are thermally welded along the shapes of the peripheral edge portions of the four sides of the air bag 101 to constitute the air bag 101 of the fourth layer (the fourth layer) from the wrist 200 side.

As illustrated in FIGS. 12 and 13, the fourteenth sheet member 106h and the fifteenth sheet member 106i are disposed facing each other, and include a plurality of openings 106h1, 106i1, respectively, through which the two air bags 101 are fluidly continuous. The fifteenth sheet member 106i and the sixteenth sheet member 106j are thermally welded along the shapes of the peripheral edge portions of the four sides of the air bag 101 to constitute the air bag 101 of the fifth layer (the fifth layer) from the wrist 200 side.

Note that the thirteenth sheet member 106g, the fourteenth sheet member 106h, the fifteenth sheet member 106i, and the sixteenth sheet member 106j are integrally and thermally welded along the shapes of the peripheral edge portions of the four sides of the air bags 101 to constitute the second intermediate layer 113 in which the air bags 101 of the fourth layer and the fifth layer are integrally formed.

As illustrated in FIGS. 12 and 13, the sixteenth sheet member 106j and the seventeenth sheet member 106k are disposed facing each other, and include a plurality of openings 106j1, 106k1, respectively, through which the two air bags 101 are fluidly continuous. The seventeenth sheet member 106k is configured in a shape so as to ensure configuring the air bag 101 and the joined portions 102, for example. The sixteenth sheet member 106j and the seventeenth sheet member 106k are integrally joined by bridge welding by heat on the peripheries of the plurality of openings 106j1, 106k1 in a quadrilateral frame shape smaller than the four sides where the air bag 101 is welded.

The seventeenth sheet member 106k and the eighteenth sheet member 106l are thermally welded along the shapes of the peripheral edge portions of the four sides of the air bag 101 and cut into predetermined shapes to constitute the air bag 101 of the sixth layer (the sixth layer) from the wrist 200 side including the notch portions 104 and the joined portions 102.

Furthermore, the eighteenth sheet member 106l has a hole portion 106l1 into which the distal end of the connection portion 103 can be inserted, for example. The connection portion 103 is disposed in the hole portion 106l1 of the eighteenth sheet member 106l, and the periphery of the hole portion 106l1 is thermally welded with the connection portion 103. Further, the eighteenth sheet member 106l is bonded to the inner circumferential surface of the curler 5 and the joined portions 102 of the seventeenth sheet member 106k are each bonded to the outer circumferential surface of the curler 5 via the bonding layer 75.

Additionally, each of the sheet members 86, 96, and 106 forming the pressing cuff 71, the sensing cuff 73, and the tensile cuff 74 are made of a thermoplastic resin material. The thermoplastic resin material is a thermoplastic elastomer. Examples of thermoplastic resin material constituting the sheet members 86, 96, and 106 include thermoplastic polyurethane based resin (hereinafter referred to as TPU), polyvinyl chloride resin, ethylene-vinyl acetate resin, thermoplastic polystyrene based resin, thermoplastic polyolefin resin, thermoplastic polyester based resin, and thermoplastic polyamide resin. Note that, in the pressing cuff 71 and the sensing cuff 73, among at least the plurality of sheet members 86 and 106 constituting the air bags 81 and 101, at least the sheet members 86 and 106 welded to the curler 5 are made of a material same kind of the material of the curler 5.

For example, the sheet members 86, 96, and 106 are formed using a molding method such as T-die extrusion molding or injection molding. After being molded by each molding method, the sheet members 86, 96, and 106 are sized into predetermined shapes, and the sized individual pieces are joined by welding or the like to constitute bag-like structures 81, 91, and 101. A high frequency welder or laser welding is used as the welding method.

The fluid circuit 7 is constituted by the case 11, the pump 14, the flow path unit 15, the on-off valves 16, the pressure sensors 17, the pressing cuff 71, the sensing cuff 73, and the tensile cuff 74. A specific example of the fluid circuit 7 will be described below.

As illustrated in FIG. 5, the fluid circuit 7 includes, for example, a first flow path 7a in which the pump 14 and the sensing cuff 73, and the first pressure sensor 17A and the second pressure sensor 17B are continuous via the first on-off valve 16A, a second flow path 7b configured by being branched from the first flow path 7a between the pump 14 and the first on-off valve 16A in which the pump 14 and the atmosphere are continuous via the second on-off valve 16B, the third on-off valve 16C, and the fourth on-off valve 16D in order, a third flow path 7c configured by being branched from the middle portion between the second on-off valve 16B and the third on-off valve 16C of the second flow path 7b in which the tensile cuff 74 is continuous from the pump 14, and a fourth flow path 7d configured by being branched from the middle portion between the third on-off valve 16C and the fourth on-off valve 16D of the second flow path 7b in which the pressing cuff 71 is continuous from the pump 14.

In the fluid circuit 7, the second on-off valve 16B and the third on-off valve 16C are opened and the first on-off valve 16A and the fourth on-off valve 16D are closed to connect the third flow path 7c and the fourth flow path 7d branched from the second flow path 7b to the pump 14, and the pump 14, the pressing cuff 71, and the tensile cuff are fluidly connected.

In the fluid circuit 7, the first on-off valve 16A, the second on-off valve 16B, and the third on-off valve 16C are opened and the fourth on-off valve 16D is closed to connect the first flow path 7a and the third flow path 7c and the fourth flow path 7d branched from the second flow path 7b to the pump 14, and the pump 14, the pressing cuff 71, and the tensile cuff, and the pump 14 and the sensing cuff 73 are fluidly connected. In the fluid circuit 7, the second on-off valve 16B, the third on-off valve 16C, and the fourth on-off valve 16D are opened and the first on-off valve 16A is closed to connect the second flow path 7b, the third flow path 7c, and the fourth flow path 7d to the pump 14, and the pump 14, the pressing cuff 71, the tensile cuff 74, and the atmosphere are fluidly connected. In the fluid circuit 7, the first on-off valve 16A, the second on-off valve 16B, the third on-off valve 16C, and the fourth on-off valve 16D are opened to connect the first flow path 7a, the second flow path 7b, the third flow path 7c, and the fourth flow path 7d to the pump 14, and the pump 14, the pressing cuff 71, the sensing cuff 73, the tensile cuff 74, and the atmosphere are fluidly connected.

As illustrated in FIGS. 2, 6, and 9, the power feed unit 8 is provided in the recess 5c formed in the outer surface on the first end side of the curler 5 protruding from the device main body 3. For example, the power feed unit 8 is configured to be connectable to a connector provided on a charging cable of a charger.

As illustrated in FIGS. 2, 6, and 9, the power feed unit 8 includes a wiring portion 8a, the power feed terminals 8b, and a cover 8c that covers the wiring portion 8a disposed in the recess 5c of the curler 5. The wiring portion 8a has a first end connected to the power feed terminals 8b and the second end connected to the control unit 55. The power feed terminals 8b are constituted by two circular terminals, for example. For example, the wiring portion 8a and the power feed terminals 8b are formed by, for example, flexible printed circuits (FPC) in which, for example, a conductive metal film is provided on a base film, such as polyimide. The cover 8c is formed in the same shape as the recess 5c, covers the recess 5c, and when provided in the recess 5c, its upper surface is flush with the outer surface on the short side of the curler 5.

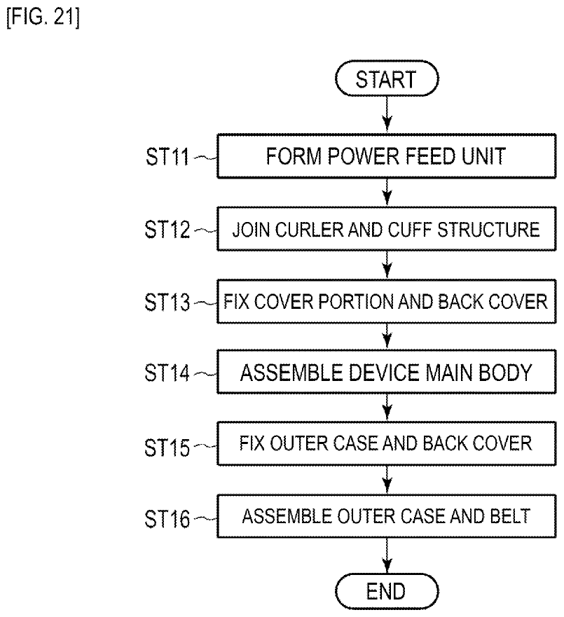

Next, an example of a manufacturing method of the blood pressure measurement device 1 will be described below using FIG. 21.

First, the power feed unit 8 is formed in the curler 5 (step ST11). The FPC constituting the wiring portion 8a and the power feed terminals 8b in the curler 5 are bonded to the cover portion 5a and the recess 5c of the curler 5 with, for example, a double-sided tape, and the cover 8c is bonded to the recess 5c with, for example, a double-sided tape.

Next, the cuff structure 6 is joined to the curler 5 (step ST12). As a specific example, first, the back plate 72 is disposed in a curved jig, and the back plate 72 is overheated by a heating furnace for a thermal process to curve the back plate 72 in a predetermined shape. Next, the double-sided tapes as the bonding layers 75 are stuck to a region facing the curler 5 of the fourth sheet member 86d of the pressing cuff 71 and the joined portions 82 to stick the pressing cuff 71 to the curler 5. Next, the double-sided tape is stuck to a region facing the back plate 72 of the sixth sheet member 96b of the sensing cuff 73 to stick the sensing cuff 73 to the back plate 72. Note that in these steps, the connection portion 84 of the pressing cuff 71 and the connection portion 93 of the sensing cuff 73 are inserted through the first hole portion 5f1 and the second hole portion 5f2 in the cover portion 5a of the curler 5.

Next, the double-sided tape is stuck to a region facing the pressing cuff 71 of the back plate 72 to stick the back plate 72 to the first sheet member 86a of the pressing cuff 71. Next, double-sided tapes are stuck to a region facing the curler 5 of the eighteenth sheet member 106l and the joined portions 102 of the tensile cuff 74 to stick the tensile cuff 74 to the curler 5 and the flow path body 83 of the pressing cuff 71 and the flow path body 92 of the sensing cuff 73 disposed on the inner surface of the curler 5. Through these steps, the cuff structure 6 is bonded to the curler 5.

Next, the sealing member 36 and the back cover 35 are disposed on the cover portion 5a, and the back cover 35 is fixed to the cover portion 5a with the first fastening members 35a (step ST13) to constitute the back lid.

Next, the device main body 3 except for the back cover 35 is integrally assembled (step ST14). Next, the back cover 35 is disposed on an end portion on the wrist 200 side of the outer case 31 of the device main body 3, and the outer case 31 and the back cover 35 are fixed with the second fastening members 35b (step ST15). Then, the first belt 61 and the second belt 62 are assembled to the outer case 31 (step ST46). Through these steps, the blood pressure measurement device 1 is manufactured.

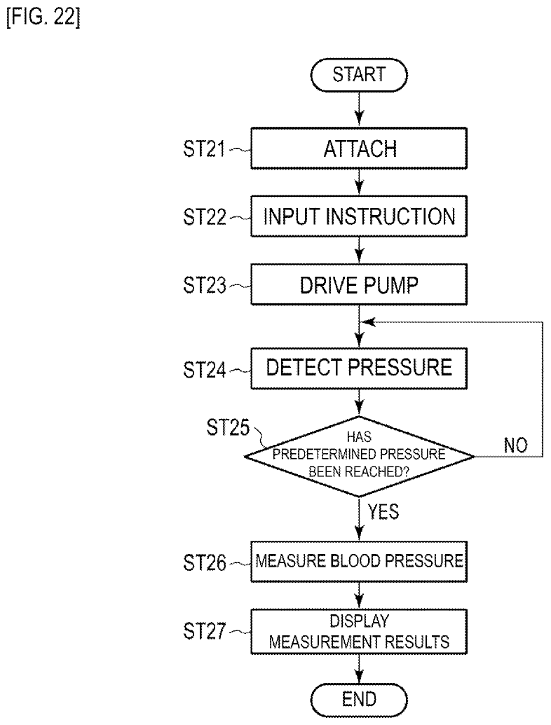

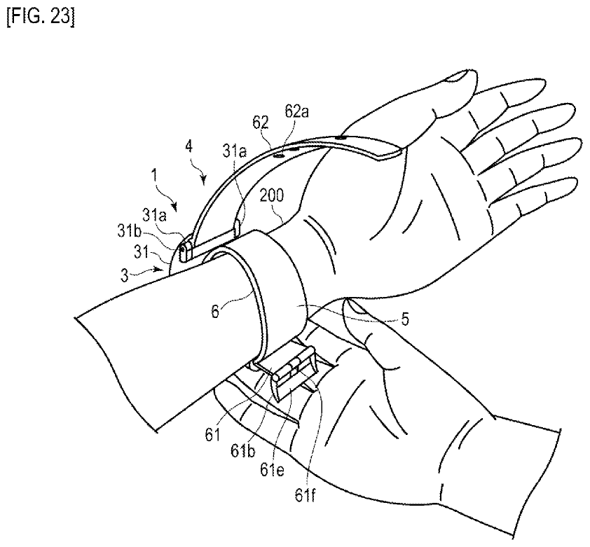

Next, an example of measurement of a blood pressure value using the blood pressure measurement device 1 will be described using FIGS. 22 to 25. FIG. 22 is a flowchart depicting an example of a blood pressure measurement using the blood pressure measurement device 1, depicting both of an operation by a user and an operation by the control unit 55. Additionally, FIGS. 23 to 25 illustrate an example of the user wearing the blood pressure measurement device 1 on the wrist 200.

First, the user attaches the blood pressure measurement device 1 to the wrist 200 (step ST21). As a specific example, as illustrated in FIG. 23, for example, the user inserts one of the wrists 200 into the curler 5.

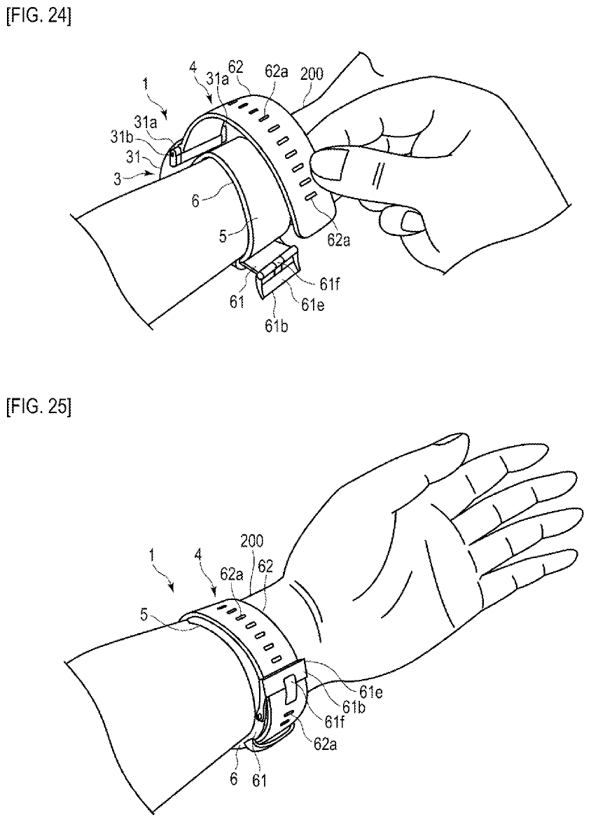

At this time, in the blood pressure measurement device 1, the device main body 3 and the sensing cuff 73 are disposed at opposite positions in the curler 5, and thus the sensing cuff 73 is disposed in a region on the hand palm side of the wrist 200 where the artery 210 is present. Thus, the device main body 3 and the tensile cuff 74 are disposed on the hand back side of the wrist 200. Then, as illustrated in FIG. 24, the user passes the second belt 62 through the frame body 61e of the buckle 61b of the first belt 61 with the hand opposite to the hand on which the blood pressure measurement device 1 is disposed. Next, the user then pulls the second belt 62 to bring the member on the inner circumferential surface side of the curler 5, that is, the cuff structure 6, into close contact with the wrist 200, and inserts the prong 61f into the small hole 62a. Thus, as illustrated in FIGS. 4 and 26, the first belt 61 and the second belt 62 are connected, and the blood pressure measurement device 1 is attached to the wrist 200.

Then, the user operates the operation unit 13 to input an instruction corresponding to the start of measurement of the blood pressure value. The operation unit 13, on which an input operation of the instruction has been performed, outputs an electrical signal corresponding to the start of the measurement to the control unit 55 (step ST22). The control unit 55 receives the electrical signal, and then, for example, opens the first on-off valve 16A, the second on-off valve 16B, and the third on-off valve 16C and closes the fourth on-off valve 16D, and drives the pump 14 to feed compressed air to the pressing cuff 71, the sensing cuff 73, and the tensile cuff 74 through the first flow path 7a, the second flow path 7b, the third flow path 7c, and the fourth flow path 7d (step ST23). Thus, the pressing cuff 71, the sensing cuff 73, and the tensile cuff 74 start to be inflated.

The first pressure sensor 17A and the second pressure sensor 17B detect the pressures in the pressing cuff 71, the sensing cuff 73, and the tensile cuff 74, and output, to the control unit 55, electrical signals corresponding to the pressures (step ST24). Based on the received electrical signals, the control unit 55 determines whether the pressures in the internal spaces of the pressing cuff 71, the sensing cuff 73, and the tensile cuff 74 have reached a predetermined pressure for measurement of the blood pressure (step ST25). For example, in a case where the internal pressures of the pressing cuff 71 and the tensile cuff 74 have not reached the predetermined pressure and the internal pressure of the sensing cuff 73 has reached the predetermined pressure, the control unit 55 closes the first on-off valve 16A and feeds compressed air through the first flow path 7a, the second flow path 7b, the third flow path 7c, and the fourth flow path 7d.

When the internal pressures of the pressing cuff 71 and the tensile cuff 74 and the internal pressure of the sensing cuff 73 all have reached the predetermined pressure, the control unit 55 stops driving the pump 14 (YES in step ST25). At this time, as indicated by the two-dot chain line in FIG. 4, the pressing cuff 71 and the tensile cuff 74 are sufficiently inflated, and the inflated pressing cuff 71 presses the back plate 72. Additionally, the tensile cuff 74 presses against the curler 5 in a direction away from the wrist 200, and then the belt 4, the curler 5, and the device main body 3 move in a direction away from the wrist 200, and as a result, the pressing cuff 71, the back plate 72, and the sensing cuff 73 are pulled toward the wrist 200 side. In addition, when the belt 4, the curler 5, and the device main body 3 move in the direction away from the wrist 200 due to the inflation of the tensile cuff 74, the belt 4 and the curler 5 move toward both lateral sides of the wrist 200, and the belt 4, the curler 5, and the device main body 3 move in a state of close contact with both lateral sides of the wrist 200. Thus, the belt 4 and the curler 5, which are in close contact with the skin of the wrist 200, pull the skin on both lateral sides of the wrist 200 toward the hand back side. Note that the curler 5 may be configured to indirectly contact the skin of the wrist 200 with the sheet members 86 or 106 in between, for example, as long as the curler 5 can pull the skin of the wrist 200.

Further, the sensing cuff 73 is inflated by being fed with a predetermined amount of air such that the internal pressure equals the pressure required to measure blood pressure, and is pressed toward the wrist 200 by the back plate 72 that is pressed by the pressing cuff 71. Thus, the sensing cuff 73 presses the artery 210 in the wrist 200 and occludes the artery 210 as illustrated in FIG. 26.

Additionally, the control unit 55, for example, controls the third on-off valve 16C and repeats the opening and closing of the third on-off valve 16C, or adjusts the degree of opening of the third on-off valve 16C to pressurize the pressure of the internal space of the pressing cuff 71. In the process of pressurization, based on the electrical signal output by the second pressure sensor 17B, the control unit 55 obtains measurement results such as blood pressure values, for example, the systolic blood pressure and the diastolic blood pressure, and the heart rate and the like (step ST26). The control unit 55 outputs an image signal corresponding to the obtained measurement results to the display unit 12, and displays the measurement results on the display unit 12 (step ST27). In addition, after the end of the blood pressure measurement, the control unit 55 opens the first on-off valve 16A, the second on-off valve 16B, the third on-off valve 16C, and the fourth on-off valve 16D.

The display unit 12 receives the image signal, and then displays the measurement results on the screen. The user views the display unit 12 to confirm the measurement results. After the measurement is complete, the user removes the prong 61f from the small hole 62a, removes the second belt 62 from the frame body 61e, and removes the wrist 200 from the curler 5, thus removing the blood pressure measurement device 1 from the wrist 200.

The blood pressure measurement device 1 according to one embodiment configured in this manner includes the cover portion 5a in a portion of the curler 5, and the cover portion 5a is integrated with the back cover 35, thereby constituting the back cover covering the opening on the wrist 200 side of the outer case 31. Additionally, the back cover 35 is connected to the outer case 31 and the cover portion 5a with the first fastening members 35a and the second fastening members 35b. With the configuration, the curler 5 and the outer case 31 can be fixed via the back cover 35 constituting a part of the back cover, and this eliminates the need for providing, for example, a hook or a claw in the curler and disposing, for example, a hook or a claw inside the device main body 3. Since this allows a reduction in dimensions in the thickness direction of the device main body 3, the blood pressure measurement device 1 can be downsized. Additionally, this eliminates the need for providing a hole for a hook or a claw, thereby ensuring securing the waterproof property of the device main body 3 and securing strength of the cover portion 5a.

Additionally, this eliminates the need for providing a hole for inserting, for example, a hook or a claw in the cuff structure 6, this improves a degree of freedom of design to dispose the connection portions 84, 93, 103 provided on the respective cuffs 71, 73, 74 of the cuff structure 6. Therefore, the second hole portion 5f2 through which the connection portion 93 of the sensing cuff 73 is inserted is easily designed to be disposed on the second end side of the cover portion 5a. As a result, the length of the second flow path body 92 that connects the air bag 91 of the sensing cuff 73 and the flow path unit 15 of the device main body 3 can be shorter than the length of the first flow path body 83 that connects the air bag 81 of the pressing cuff 71 and the flow path unit 15 of the device main body 3. This configuration allows reducing the region other than the air bag 91 for sensing in the sensing cuff 73, that is, a volume of the flow path body 92, as much as possible even in the configuration in which the device main body 3 is disposed on the hand back side of the wrist 200 and the sensing cuff 73 is disposed on the hand palm side of the wrist 200. This allows the blood pressure measurement device 1 to suitably detect the blood pressure in blood pressure measurement by the sensing cuff 73, thereby ensuring improving blood pressure measurement accuracy.

Providing the insert member 5d for reinforcement on the cover portion 5a of the curler 5 constituting the back lid together with the back cover 35 allows reducing the thickness of the cover portion 5a while ensuring improving the strength, compared to a case where the cover portion 5a is made of only with a resin material without the insert member 5d. Additionally, by reinforcing the cover portion 5a with the insert member 5d made of a metal material having higher strength than the resin material that forms the curler 5, the thickness of the cover portion including the insert member 5d can be reduced to the utmost extent, and the joining strength with the first fastening members 35a can be improved. As a result, since the thickness of the cover portion 5a can be thinned, the blood pressure measurement device 1 can be downsized.

Additionally, by fixing the back cover 35 to the cover portion 5a and the outer case 31 with the first fastening members 35a and the second fastening members 35b, respectively, the back cover 35 and the outer case 31, and the back cover 35 and cover portion 5a can be easily fixed and disassembled. This facilitates manufacturing, replacement of the component, and maintenance of the blood pressure measurement device 1. Moreover, since the cover portion 5a includes the insert member 5d for reinforcement, the joining strength of the first fastening members 35a to the cover portion 5a can be improved, and the first fastening members 35a can be downsized. As a result, the inner diameters of the hole portions 35c in the back cover 35 and the screw holes 5e in the cover portion 5a can be reduced, thereby downsizing the device main body 3 and improving the degree of freedom of the design of the device main body 3.

Furthermore, by providing the relief portions 5b at the positions facing the second fastening members 35b, which fix the back cover 35 and the outer case 31, the outer case 31 can be fixed to the back cover 35 using the second fastening members 35b with the back cover 35 attached to the cover portion 5a of the curler 5. This facilitates assembly and disassembly of the blood pressure measurement device 1. Note that when the tensile cuff 74 covers the relief portions 5b of the curler 5, it is only necessary that the bonding layer 75 is not provided between the position of covering the relief portions 5b and peripheries of the relief portions 5b and the end edge of the curler 5 close to the relief portions 5b of the tensile cuff 74. With this configuration, bending the tensile cuff 74 allows releasing both main surfaces of the curler 5 and disposing, for example, the second fastening members 35b and tools in the relief portions 5b.

Additionally, since the second fastening members 35b can be provided at the positions facing the curler 5, the degree of freedom of arrangement of the second fastening members 35b is improved. For example, as in the present embodiment, providing the screw holes 31c at the four locations on the bases of the lugs 31a of the outer case 31 eliminates the need for providing protrusions to provide the screw holes 31c in the outer case 31. In addition, the screw holes 31c are disposed in the rear surface of the lugs 31a, and therefore protrusions to provide the hole portions 35d of the back cover 35 are covered with the lugs 31a. This allows the blood pressure measurement device 1 to improve designability.

The tensile cuff 74 including the plurality of, six layers in the present embodiment, bag-like structures 101 is disposed on the hand back side of the wrist 200 including the region where the cover portion 5a of the curler 5 is provided. Thus, since an amount of inflation becomes the largest here among the respective configurations of the cuff structure 6, when the cuff structure 6 is inflated after the blood pressure measurement device 1 is attached to the wrist 200, a force applied to the curler 5 by the cuff structure 6 becomes large on the hand back side of the wrist 200 of the curler 5. However, since the insert member 5d for reinforcement is disposed on the cover portion 5a of the present embodiment, even when an external force is applied to the hand back side of the wrist 200 of the curler 5 by expansion of the tensile cuff 74, strength of the curler 5 is improved by the cover portion 5a. As a result, deformation of the curler 5 due to the expansion of the tensile cuff 74 and creep deformation caused by repeated use can be suppressed. In addition, since the creep deformation can be suppressed, the curler 5 can maintain a predetermined shape even when the blood pressure measurement device 1 is repeatedly used, and therefore the blood pressure measurement device 1 can suppress a decrease in measurement accuracy caused in blood pressure measurements.

Furthermore, by configuring the mutual abutment surfaces of the back cover 35 and the cover portion 5a to be flat surfaces and providing the sealing member 36 between the abutment surfaces, the waterproof property between the back cover 35 and the cover portion 5a can be improved in a simple configuration.

As described above, with the blood pressure measurement device 1 according to the present embodiment, the cover portion 5a including the insert member 5d and the outer case 31 are connected with the back cover 35, thereby ensuring downsizing.

Note that the present invention is not limited to the embodiments described above. In the example described above, the configuration in which the pressing cuff 71 and the sensing cuff 73 include the air bags 81, 91, the flow path bodies 83, 92, and the connection portions 84, 93 has been described, but the present invention is not limited to the configuration. For example, the pressing cuff 71 and the sensing cuff 73 may be configured to further include a member that generates a clearance between at least the two sheet members 86, 96 constituting inside of the flow path bodies 83, 92. For example, as such a member, a thread-like member, such as a nylon yarn, can be applied.

That is, the present invention is not limited to the embodiments, and various modifications can be made in an implementation stage without departing from the gist. Furthermore, each of the embodiments may be combined as appropriate to obtain the combined effects of the embodiments. Also, the embodiments described above include various stages of invention, and various inventions may be obtained by appropriately combining the multiple configuration requirements disclosed.

### REFERENCE SIGNS LIST

1 Blood pressure measurement device
3 Device main body
4 Belt
5 Curler
5a Cover portion
5b Relief portion
5c Recess
5d Insert member
5e Screw hole
5f Hole portion
5f1 First hole portion
5f2 Second hole portion
5f3 Third hole portion
6 Cuff structure
7 Fluid circuit
7a First flow path
7b Second flow path
7c Third flow path
7d Fourth flow path
8 Power feed unit
8a Wiring portion
8b Power feed terminal
8c Cover
11 Case
12 Display unit
13 Operation unit
14 Pump
15 Flow path unit
16 On-off valve
16A First on-off valve
16B Second on-off valve
16C Third on-off valve
16D Fourth on-off valve
17 Pressure sensor
17A First pressure sensor
17B Second pressure sensor
18 Power supply unit
19 Vibration motor
20 Control substrate
31 Outer case
31a Lug
31b Spring rod
31c Screw hole
32 Windshield
33 Base
35 Back cover
35a First fastening member
35b Second fastening member
35c Hole portion
35d Hole portion
36 Sealing member
41 Button
42 Sensor
43 Touch panel
51 Substrate
52 Acceleration sensor
53 Communication unit
54 Storage unit
55 Control unit
56 Main CPU
57 Sub-CPU
61 First belt
61a Belt portion
61b Buckle
61c First hole portion
61d Second hole portion
61e Frame body
61f Prong
62 Second belt
62a Small hole
62b Third hole portion
71 Pressing cuff (cuff)
72 Back plate
72a Groove
73 Sensing cuff (cuff)
74 Tensile cuff (cuff)
75 Bonding layer
81 Air bag (bag-like structure, first bag-like structure)
81a Welded portion
81b Bridge welded portion
82 Joined portion
83 Flow path body (first flow path body)
83a Welded portion
84 Connection portion (first connection portion)
86 Sheet member
86a First sheet member (sheet member)
86b Second sheet member (sheet member)
86b1 Opening
86c Third sheet member
86c1 Opening
86d Fourth sheet member
86d1 Hole portion
91 Air bag (bag-like structure, second bag-like structure)
91a Welded portion
91b Bonding margin
92 Flow path body (second flow path body)
92a Welded portion
93 Connection portion (second connection portion)
96 Sheet member
96a Fifth sheet member
96b Sixth sheet member
96b1 Hole portion
101 Air bag (bag-like structure, third bag-like structure)
101a Welded portion
101b Bridge welded portion
102 Joined portion
102a Relief portion
103 Connection portion (third connection portion)
104 Notch portion
106 Sheet member
106a Seventh sheet member
106b Eighth sheet member
106b1 Opening
106c Ninth sheet member
106c1 Opening
106d Tenth sheet member
106d1 Opening
106e Eleventh sheet member
106e1 Opening
106f Twelfth sheet member
106f1 Opening
106 g Thirteenth sheet member
106g1 Opening
106 h Fourteenth sheet member
106h1 Opening
106i Fifteenth sheet member
106i1 Opening
106j Sixteenth sheet member
106j1 Opening
106k Seventeenth sheet member
106k1 Opening
106l Eighteenth sheet member
106l1 Hole portion
111 First outer layer
112 First intermediate layer
113 Second intermediate layer
114 Second outer layer
200 Wrist
210 Artery

1. A blood pressure measurement device attached to a wrist comprising:
a curler that curves following a circumferential direction of the wrist from a hand back side of the wrist to a region where at least an artery is present on a hand palm side of the wrist through a first lateral side of the wrist, the curler including a cover portion formed on the hand back side of the wrist and an insert member for reinforcement inserted into the cover portion, the curler being made of a resin material; and
a device main body that includes an outer case and a back cover, the outer case internally including a pump and a flow path unit that supplies a fluid from the pump to a secondary side, the back cover being fixed to a first end portion of the outer case and the cover portion, a part of the back cover facing the cover portion being open, the back cover covering the first end portion of the outer case together with the cover portion.

2. The blood pressure measurement device according to claim 1, comprising:
a first fastening member that fixes the back cover to the cover portion; and
a second fastening member that fixes the outer case and the back cover.

3. The blood pressure measurement device according to claim 2, wherein
the curler includes a relief portion through which the second fastening member is insertable at a position facing the second fastening member.

4. The blood pressure measurement device according to claim 1, further comprising:
a pressing cuff that includes a plurality of first bag-like structures, a first flow path body, and a first connection portion, the plurality of first bag-like structures being formed by joining two sheet members made of a resin material, the plurality of first bag-like structures being fixed to an inner circumferential surface on the hand palm side of the wrist of the curler and inflated by the fluid supplied from the pump, the first flow path body being integrally formed with the first bag-like structure by joining the two sheet members forming the first bag-like structure facing the curler, the first flow path body fluidly connecting the pump and the first bag-like structure, the first flow path body having a distal end disposed on the wrist side of the cover portion, the first connection portion being provided at the distal end of the first flow path body, the first connection portion being inserted through the cover portion to be connected to the flow path unit;
a sensing cuff that includes a second bag-like structure, a second flow path body, and a second connection portion, the second bag-like structure being formed by joining the two sheet members, the second bag-like structure being fixed to a main surface on the wrist side of a back plate and inflated by the fluid supplied from the pump, the second flow path body being integrally formed with the second bag-like structure by joining the two sheet members forming the second bag-like structure, the second flow path body fluidly connecting the pump and the second bag-like structure, the second flow path body having a distal end disposed on the wrist side of the cover portion, the second connection portion being provided at the distal end of the second flow path body, the second connection portion being inserted through the cover portion to be connected to the flow path unit; and
a tensile cuff that includes a plurality of third bag-like structures and a third connection portion, the plurality of third bag-like structures being formed by joining the two sheet members, the plurality of third bag-like structures being fixed to an inner circumferential surface on the hand back side of the wrist of the curler and a position facing the cover portion and inflated by the fluid supplied from the pump, the third connection portion being provided at third bag-like structure facing the curler, the third connection portion being inserted through the cover portion to be connected to the flow path unit, wherein
the cover portion has respective three hole portions through which the first connection portion, the second connection portion, and the third connection portion are inserted.

5. The blood pressure measurement device according to claim 4, wherein
the hole portion through which the second connection portion is inserted is disposed on a side where the sensing cuff of the cover portion is disposed with respect to the hole portions through which the first connection portion and the third connection portion are inserted.

6. The blood pressure measurement device according to claim 1, wherein
the insert member is made of a metal material.

7. The blood pressure measurement device according to claim 1, wherein
the back cover and the cover portion mutually have flat abutment surfaces, and
a sealing member is disposed between the back cover and the cover portion.
